(19) United States
(12) Patent Application Publication
(10) Pub. No.: US 2023/0358750 A1
(43) Pub. Date: Nov. 9, 2023

(54) PROGNOSTIC VALUE OF BIOMARKERS IN PATIENTS WITH NON-SMALL CELL LUNG CANCER HAVING STABLE DISEASE

(71) Applicant: Roche Diagnostics Operations, Inc., Indianapolis, IN (US)

(72) Inventors: Anika Mang, Holzkirchen (DE); Thomas Muley, Heidelberg (DE); Vinzent Rolny, München (DE); Birgit Wehnl, Muenchen (DE)

(21) Appl. No.: 18/352,183

(22) Filed: Jul. 13, 2023

Related U.S. Application Data
(63) Continuation of application No. PCT/EP2022/050507, filed on Jan. 12, 2022.

(30) Foreign Application Priority Data
Jan. 14, 2021 (EP) 21151645.5

(51) Int. Cl. G01N 33/574

(52) U.S. Cl. CPC G01N 33/57423; G01N 2333/4742; G01N 2333/4725; G01N 2333/70596

(57) ABSTRACT

The present invention relates to an in vitro method for assessing the risk of non-small cell lung carcinoma (NSCLC) disease progression for a subject classified to have stable disease under an ongoing NSCLC treatment regime. The method involves determining the level of CYFRA 21-1 and/or the level of CA 125 in a sample obtained from the subject; and comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score. The method of the invention further allows for assessing whether the subject responds to the ongoing treatment and/or whether the treatment regime should be maintained or modified. The invention also provides for corresponding uses, computer-implemented methods and computer program products.

### FIELD OF THE INVENTION

The present invention relates to an in vitro method for assessing the risk of non-small cell lung carcinoma (NSCLC) disease progression for a subject classified to have stable disease while an ongoing NSCLC treatment regime. The method involves determining the level of CYFRA 21-1 and/or the level of CA 125 in a sample obtained from the subject; and comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score. The method of the invention further allows for assessing whether the subject responds to the ongoing treatment and/or whether the treatment regime should be maintained or modified. Also provided herein are corresponding uses, computer-implemented methods and computer program products.

### BACKGROUND OF THE INVENTION

Lung cancer has the highest incidence among all cancer types in the world (over 2 million new cases were estimated in 2018) and second highest incidence in Central and Eastern Europe (GLOBOCAN 2018, 2018 ed.; International Agency for Research on Cancer. World Health Organization: 2018). It is also the leading cause of cancer mortality, accounting for 131,000 deaths per year in Central and Eastern Europe and 1.8 million deaths per year worldwide (GLOBOCAN 2018, 2018 ed.; International Agency for Research on Cancer. World Health Organization: 2018).

Approximately 85% of all cases of lung cancer are non-small cell lung cancer (NSCLC). The most common histological subtypes of NSCLC are adenocarcinoma (ADC; 40% of all cases) and squamous cell carcinoma (SCC; 35% of all cases) (Bender, E., Nature 2014, 513, S2-S3, doi:10.1038/513S2a).

Lung cancer is largely asymptomatic, and >70% of all cases are diagnosed at late stages (Molina, J. R. et al., Mayo Clinic Proceedings 2008, 83, 584-594, doi:10.4065/83.5.584). Treatment options include surgery, radiation therapy, chemotherapy and immune checkpoint inhibitors; adjuvant chemotherapy is recommended for patients with Stage II and IIIA NSCLC (Postmus, P. E. et al., Annals of Oncology 2017, 28, iv1-iv21, doi:10.1093/annonc/mdx222; NCCN Guidelines. Non-Small-Cell Lung Cancer. Version 4.2019). Additionally, small-molecule tyrosine kinase inhibitors have been shown to improve survival and are recommended for patients with advanced stage NSCLC who have activating mutations in epidermal growth factor receptor, anaplastic lymphoma kinase. ROS1 or BRAF (NCCN Guidelines. Non-Small-Cell Lung Cancer, Version 4.2019; Planchard, D. et al., Annals of Oncology 2018, 29, iv192-iv237, doi:10.1093/annonc/mdy275).

Computed tomography (CT) or positron emission tomography (PET) are widely used for clinical staging (Molina, J. R. et al., Mayo Clinic Proceedings 2008, 83, 584-594. doi:10.4065/83.5.584; Postmus, P. E. et al., Annals of Oncology 2017, 28, iv1iv21, doi:10.1093/annonc/mdx222; NCCN Guidelines. Non-Small-Cell Lung Cancer. Version 4.2019). Moreover, response criteria based on CT imaging results have been successfully used in numerous randomized clinical trials, and CT is currently the method of choice for assessing treatment response (Eisenhauer, E. A. et al., Eur J Cancer 2009, 45, 228-247, doi:10.1016/j.ejca.2008.10.026).

CT-based response criteria were developed in patients receiving cytotoxic chemotherapy, as a means of evaluating active agents based on the amount of tumor shrinkage they caused. WHO and later RECIST criteria were adopted as the gold standard for assessing treatment response in clinical trials and bi-lateral and uni-lateral measurements remain the mainstay of response assessment in routine clinical practice. Nevertheless, objective tumor response assessed based on imaging shows imperfect correlation with patients outcomes, and its utility has been challenged in recent studies assessing non-chemotherapeutic treatment (Lee H. Y et al., Lung Cancer 2011, 73, 63-69; Hwang K and Kim H, Tuberc Respir Dis (Seoul) 2017, 80, 136-142, doi:10.4046/trd.2017.80.2.136), particularly with checkpoint inhibitors (M. Tazdait et al., European Journal of Cancer, Volume 88, 2018, Pages 38-47; Wolchok. J D et al., Clin Cancer Res Dec. 1 2009 (15) (23) 7412-7420).

A number of studies in patients with lung cancer have demonstrated that cancer biomarkers, such as CYFRA 21-1, CFA. ProGRP and NSE, can be used to determine lung cancer subtype, and correlate with tumor stage, prognosis and response to therapy (Jing A. G. et al., BMC Cancer 2015, 15, doi:10.1186/s12885-015-1403-x; Liu, L et al., BioMed Research International 2017, 2017, 1-9, doi:10.1155/2017/2013989; Muley, T. et al., Lung Cancer 2018, 120, 46-53, doi-10.1016/j.lungcan 2018.03.015; Wojcik E and Kulpa J K, Lung Cancer (Auck1) 2017; 8:231-40). However, compelling use cases demonstrating an additional value of biomarkers vis-a-vis imaging approaches are currently lacking. In particular, it is largely unclear whether combining imaging and biomarker assessment provides additional value for guiding therapy or prognosis.

The assessment of therapeutic efficacy in advanced non-small cell lung cancer (NSCLC) is currently based on imaging by computed tomography (CT) scanning. Established definitions exist for therapy response evaluation of solid tumors (RECIST) which classify the response into four main categories of complete response (CR), partial response (PR), stable disease (SD) and progressive disease (PD) depending on the reduction/increase in tumor volume, number of lesions and new lesions/metastases (New response evaluation criteria in solid tumours: Revised RECIST guideline (version 1.1) E. A. Eisenhauer et al, European Journal of Cancer 45 (2009) 228-247). Decision of continuation (or stop) of therapy is typically based on these imaging results and defined in treatment guidelines applied worldwide (NCCN Guidelines. Non-Small-Cell Lung Cancer. Version 4.2019, 2019. Postmus P E, Kerr K M, Oudkerk M, Senan S. Waller D A, Vansteenkiste J, et al. Early and locally advanced non-small-cell lung cancer (NSCLC): ESMO Clinical Practice Guidelines for diagnosis, treatment and follow-up^{†}. Annals of Oncology 2017; 28(suppl. 4)).

In the response categories of partial response (PR) and stable disease (SD) usually therapy is continued according to the guideline recommendation. SD group of patients include according to RECIST 1.1 criteria patients with measurable lesions that have neither sufficient tumor shrinkage to qualify as partial response (≥30% decrease in size), nor sufficient growth to qualify as a progressive disease ((≥20% increase in size). Especially in the SD group, which covers a wide range of tumor size ranges, there remains a high uncertainty with respect to the response to an ongoing treatment regime and prognosis. Yet, there are currently no reliable means available for further characterizing treatment efficacy and/or prognosis of disease progression in this group of SD patients.

Wang and coworkers (Wang t al., 2002, Tumor, (25 Dec. 2012) Vol. 32, No. 12, pp. 1021-1024) explored the clinical value of expression levels of serum tumor markers in monitoring the response to the targeted therapy with EGFR-TK1 (epidermal growth factor receptor-tyrosine kinase inhibitor) for patients with advanced NSCLC. Yet, this study did not specifically address the prognosis of disease progression in the group of SD patients by serum biomarkers. Moreover, this study focused on changes of biomarker levels only, which has the disadvantage of at least two measurements using the same assay at distinct timepoints being required.

Hall C. and coworkers (Hall C et al., Journal of Thoracic Oncology, (June 2011) Vol. 6. No. 6. Supp. SUPPL. 2, pp. S979-S980) retrospectively assessed the performance of a decrease in CYFRA 21-1 serum levels for the detection of disease progression in lung cancer patients. However, this work did not challenge the improvement of prognosis of treatment response in SD patients and focused on the measurement of CYFRA 21-1 levels over time.

A study of Iqbal Kashif (Journal of Thoracic Oncology, (June 2011) Vol. 6, No. 6, Supp. SUPPL. 2, pp. S1248-S1249) reported CEA and CYFRA 21-1 as an early predictor for the response to first line chemotherapy in advanced NSCLC. However, again this study was based on a cumbersome assessment of biomarker level changes and did not report a specific benefit for the group of patients categorized as SD by CT imaging.

Essink A. and coworkers (Essink et al., Journal of Thoracic Oncology, (April 2016) Vol. 11. No. 4, Supp. SUPPL. 1, pp. S126) studied serum tumor marker changes including CYFRA 21-1, CEA, SCC, CEA and NSE changes in the context of the response to immunotherapy treatment of NSCLC. Again, only changes in biomarker levels were assessed and no specific benefit for the group of patients categorized as SD by CT imaging was studied.

Another study assessing the reduction in CYFRA 21-1 levels upon on chemotherapy of NSCLC was conducted by Alm El-Din Mohamed A and coworkers (Alm El-Din Mohamed A et al., The international journal of biological markers, (2012 Jul. 19) Vol. 27, No. 2. pp. e139-46). This study focused on a decline of biomarker levels and, thus, also involves two or more biomarker assessments at defined timepoints. Further, this study did not report a particular benefit for assessing disease progression and/or treatment response in SD patients.

Yang Liang and coworkers (Yang Liang et al., Experimental and therapeutic medicine, (2012 August) Vol. 4, No. 2, pp. 243-248) reported declines in serum CYFRA 21-1 levels as predictor for chemotherapy response of NSCLC patients. Again only biomarker level changes were assessed and no specific benefit for SD patients was described.

Li Ling et al. investigated the value of CEA, CYFRA 21-1 as an assessment indicator of therapeutic efficacy in advanced NSCLC patients. This study again focused on changes in biomarker levels rather than absolute biomarker levels at a defined timepoint.

In view of the above, there is a high need to further improve the assessment of treatment response and prognosis, especially in NSCLC patients categorized to have a stable disease by imaging.

### SUMMARY OF THE INVENTION

The above-mentioned need is addressed by the present invention.

The present invention in particular provides for the following items:
1. An in vitro method for assessing the risk of non-small cell lung carcinoma (NSCLC) disease progression for a subject suffering from non-small cell lung carcinoma (NSCLC) and being under an ongoing NSCLC treatment regime, said method comprising:
   a) determining the level of CYFRA 21-1 and/or the level of CA 125 in a sample obtained from the subject; and
   b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score,
wherein the subject is classified to have a stable disease.
2. The method of item 1, wherein in a) the level of CYFRA 21-1 is determined, wherein the comparing in b) comprises or consists of (i) as defined in item 1b), and wherein a determined level of CYFRA 21-1 lower than or equal to the CYFRA 21-1 cut-off level is indicative for a low risk of NSCLC disease progression.
3. The method of item 1 or 2, wherein in a) the level of CYFRA 21-1 is determined, wherein the comparing in b) comprises or consists of (i) as defined in item 1b), and wherein a determined level of CYFRA 21-1 higher than the CYFRA 21-1 cut-off level is indicative for a high risk of NSCLC disease progression.
4. The method of item 2 or 3, wherein the subject suffers from NSCLC of subtype adenocarcinoma.
5. The method of item 1, wherein in a) the level of CA 125 is determined, wherein the comparing in b) comprises or consists of (ii) as defined in item 1b), and wherein a determined level of CA 125 lower than the CA 125 cut-off level is indicative for a low risk of NSCLC disease progression.
6. The method of item 1 or 5, wherein in a) the level of CA 125 is determined, wherein the comparing in b) comprises or consists of (ii) as defined in item 1b), and wherein a determined level of CA 125 higher than the CA 125 cut-off level is indicative for a high risk of NSCLC disease progression.
7. The method of item 5 or 6, wherein the subject suffers from NSCLC of subtype squamous cell carcinoma (SCC-NSCLC).
8. The method of item 1, wherein in a) the levels of CYFRA 21-1 and/or CA 125 are determined, wherein the comparing in b) comprises or consist of (iii) as defined in item 1b), and wherein a determined score lower than the cut-off score is indicative for a low risk of NSCLC disease progression.
9. The method of item for 8, wherein in a) the levels of CYFRA 21-1 and/or CA 125 are determined, wherein the comparing in b) comprises or consist of (iii) as defined in item 1b), and wherein a determined score higher than the cut-off score is indicative for a high risk of NSCLC disease progression.
10. The method of item 8 or 9, wherein in a) the level of CYFRA 21-1 and the level of CA 125 in the sample are determined and the score takes into account the determined level of CYFRA 21-1 and the determined level of CA 125.
11. The method of any one of items 10, wherein the method further comprises determining said score.
12. The method of any one of items 1 to 11, wherein the risk of NSCLC disease progression is assessed relative to the NSCLC disease progression risk of a reference population, preferably the reference population on which the respective cut-off is based.
13. The method of any one of items 1 to 12, wherein the risk of NSCLC disease progression includes the risk for tumor growth (preferably as assessed by RECIST 1.1 criteria), the risk for formation of new lesions or metastasis or and/or the risk of NSCLC caused death.
14. An in vino method for assessing whether for a subject diagnosed for non-small cell lung carcinoma (NSCLC) an ongoing NSCLC treatment regime is to be maintained or modified, said method comprising:
   a) determining the level of CYFRA 21-1 and/or the level of CA 125 in a sample obtained from the subject; and
   b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score;
wherein said subject is classified to have a stable disease.
15. The method of item 14, wherein in a) the level of CYFRA 21-1 is determined, wherein the comparing in b) comprises or consists of (i) as defined in item 14b, and wherein a determined level of CYFRA 21-1 lower than or equal to the CYFRA 21-1 cut-off level indicates that the ongoing NSCLC treatment regime is to be maintained.
16. The method of item 14 or 15, wherein in a) the level of CYFRA 21-1 is determined, wherein the comparing in b) comprises or consists of (i) as defined in item 14b), and wherein a determined level of CYFRA 21-1 higher than the CYFRA 21-1 cut-off level indicates that the ongoing NSCLC treatment regime is to be modified.
17. The method of item 15 or 16, wherein the subject suffers from NSCLC of subtype adenocarcinoma.
18. The method of item 14, wherein in a) the level of CA 125 is determined, wherein the comparing in b) comprises or consists of (ii) as defined in item 14b), and wherein a determined level of CA 125 lower than the CA 125 cut-off level indicates that the ongoing NSCLC treatment regime is to be maintained,
19. The method of item 14 or 18, wherein in a) the level of CA 125 is determined, wherein the comparing in b) comprises or consists of (ii) as defined in item 14b), and wherein a determined level of CA 125 higher than the CA 125 cut-off level indicates that the ongoing NSCLC treatment regime is to be modified.
20. The method of item 18 or 19, wherein the subject suffers from NSCLC of subtype squamous cell carcinoma (SCC-NSCLC).
21. The method of item 14, wherein in a) the levels of CYFRA 21-1 and CA 125 are determined, wherein the comparing in b) comprises or consist of (iii) as defined in item 14b), and wherein a determined score lower than the cut-off score indicates that the ongoing NSCLC treatment regime is to be maintained.
22. The method of item 14 or 21, wherein in a) the levels of CYFRA 21-1 and CA 125 are determined, wherein the comparing in b) comprises or consist of (iii) as defined in item 14b), and wherein a determined score higher than the cut-off score indicates that the ongoing NSCLC treatment regime is to be modified.
23. The method of item 21 or 22, wherein in a) the level of CYFRA 21-1 and the level of CA 125 in the sample are determined and the score takes into account the determined level of CYFRA 21-1 and the determined level of CA 125
24. The method of any one of items 21 to 23, wherein the method further comprises determining said score.
25. An in vitro method for assessing whether a subject diagnosed for non-small cell lung carcinoma (NSCLC) responds to an ongoing NSCLC treatment regime, said method comprising:
   a) determining the level of CYFRA 21-1 and/or the level of CA 125 in a sample obtained from the subject; and
   b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score, wherein said subject is classified to have a stable disease.
26. The method of item 25, wherein in a) the level of CYFRA 21-1 is determined, wherein the comparing in b) comprises or consists of (i) as defined in item 25b), and wherein a determined level of CYFRA 21-1 lower than or equal to the CYFRA 21-1 cut-off level indicates that the subject responds and/or will respond to the ongoing NSCLC treatment regime.
27. The method of item 25 or 26, wherein in a) the level of CYFRA 21-1 is determined, wherein the comparing in b) comprises or consists of (i) as defined in item 25b), and wherein a determined level of CYFRA 21-1 higher than the CYFRA 21-1 cut-off level indicates that the subject does not respond and/or will not respond to the ongoing NSCLC treatment regime.
28. The method of item 26 or 27, wherein the subject suffers from NSCLC of subtype adenocarcinoma.
29. The method of item 25, wherein in a) the level of CA 125 is determined, wherein the comparing in b) comprises or consists of (ii) as defined in item 25b), and wherein a determined level of CA 125 lower than the CA 125 cut-off level indicates that the subject responds or will respond to the ongoing NSCLC treatment regime.
30. The method of item 25 or 29, wherein in a) the level of CA 125 is determined, wherein the comparing in b) comprises or consists of (ii) as defined in item 25b), and wherein a determined level of CA 125 higher than the CA 125 cut-off level indicates that the subject does not respond or will not respond to the ongoing NSCLC treatment regime.
31. The method of item 29 or 30, wherein the subject suffers from NSCLC of subtype squamous cell carcinoma (SCC-NSCLC).
32. The method of item 25, wherein in a) the levels of CYFRA 21-1 and/or CA 125 are determined, wherein the comparing in b) comprises or consist of (iii) as defined in item 25b), and wherein a determined combination score lower than the reference indicates that the subject responds to the ongoing NSCLC treatment regime.
33. The method of item 25 or 32, wherein in a) the levels of CYFRA 21-1 and CA 125 are determined, wherein the comparing in b) comprises or consist of (iii) as defined in item 25b), and wherein a determined combination score higher than the reference indicates that the subject will not respond to the ongoing NSCLC treatment regime.
34. The method of any one of items 1 to 33, wherein the method further comprises determining the level of CEA.
35. The method of item 34, wherein the comparing in b) comprises or consists of (iii) as defined in item 1b), 14b) or 25b), respectively, and wherein the score further takes into account the determined level of CEA.
36. The method of any one of item 1 to 35, wherein the method further comprises: obtaining the information whether the subject suffers from non-small cell lung carcinoma subtype squamous cell carcinoma (SCC-NSCLC) or adenocarcinoma (ADC-NSCLC), preferably based on histological data; and wherein the comparing in b) comprises iii) as defined in item 1b). 14b) or 25b), respectively, and wherein the score further takes into account the NSCLC subtype, preferably by using an interaction term, even more preferably by using an interaction term between the information about the NSCLC subtype and the included biomarkers (i.e. CYFRA 21 and/or CA 125 and optionally CEA).
37. The method of any one of items 1 to 36, wherein the CYFRA cut-off level, the CA 125 cut-off level and/or the cut-off score is based on a reference population of subjects, preferably on a reference population comprising subjects suffering from non-small cell long carcinoma (NSCLC) that are diagnosed to have stable disease and are under the ongoing treatment regime, wherein the cut off level or score is preferably determined on the basis of the level(s) or the score after the second cycle of the treatment regime(s) has been applied to the reference population.
38. The method of any one of items 1 to 37, wherein the ongoing NSCLC treatment regime is selected from the group consisting of: chemotherapy, targeted therapy, immunotherapy and combinations thereof.
39. The method of any one of items 1 to 38, wherein said subject is a subject classified to have a stable disease based on imaging data, preferably CT or PET CT data.
40. The method of any one of items 1 to 39, wherein said subject is a subject classified to have a stable disease based on RECIST, preferably RECIST 1.1 criteria.
41. The method of any one of items 1 to 40, wherein the sample is a sample obtained 10 days to 150 days after the start date of the ongoing treatment regime, preferably 20 days to 120 days after the start date of the ongoing treatment regime, and most preferably 25 days to 108 days after the start date of the ongoing treatment regime.
42. The method of any one of items 1 to 41, wherein the subject is classified to have a stable disease based on data (e.g. tumor imaging data such as CT data) obtained 10 days to 150 days after the start date of the ongoing treatment regime, preferably 20 days to 120 days after the start date of the ongoing treatment regime and most preferably 35 days to 108 days after the start date of the ongoing treatment regime.
43. The method of any one of items 1 to 42, wherein the sample is a sample obtained after the second cycle of the treatment regime (e.g. chemotherapy), preferably wherein the sample is obtained before the third cycle of the treatment regime.
44. The method of any one of items 1 to 43, wherein the sample is a sample that was obtained within a specific time frame around the day of the medical examination (e.g. imaging such as CT) eventually leading to the subject being classified as stable disease, preferably wherein the specific time frame is from 35 days before the day of the medical examination to 35 days after the day of the medical examination, even more preferably wherein the specific time frame is from 30 days before the day of the medical examination to 30 days after the day of the medical examination and most preferably wherein the specific time frame may be from 10 days before the day of the medical examination to 29 days after the medical examination.
45. The method of any one of items 1 to 44 wherein the treatment regime is a first line treatment regime.
46. The method of any one of items 1 to 45, wherein the sample is a blood sample.
47. The method of item 46, wherein the blood sample is selected from the group consisting of whole blood, serum or plasma. 48. The method of item 46, wherein the blood sample is serum or plasma.
49. The method of any one of items 46 to 48, wherein the blood sample is venous blood sample.
50. The method of any one of items 1 to 49, wherein the method further comprises providing the information whether (i) the subject is at risk for NSCLC disease progression, (ii) the treatment regime is to be maintained or modified and/or (iii) the subject responds to an ongoing NSCLC treatment regime.
51. A method of treatment of a subject diagnosed for NSCLC, said method comprising (i) assessing whether the subject responds to an ongoing treatment regime as defined in any one of items 25 to 50 or (i)′ assessing whether an ongoing treatment regime of said subject should be maintained as defined in any one of items 14 to 24 and 34 to 50; and (ii) continuing to treat the patient with the ongoing treatment regime or modifying the treatment regime according to the result of the assessment in (i) or (i)′
52. Use of CYFRA 21-1 and/or CA 125 as biomarker(s) for assessing:
   (i) the risk of NSCLC disease progression under an ongoing NSCLC treatment regime for a subject diagnosed for NSCLC;
   (ii) whether a subject diagnosed for NSCLC responds to an ongoing NSCLC treatment regime; and/or
   (iii) whether for a subject diagnosed for NSCLC an ongoing NSCLC treatment regime is to be maintained or modified;
   wherein said subject in (i), (ii) and (iii) is classified to have a stable disease.
53. The use of item 52, further comprises using CEA as biomarker for (i), (ii) and/or (iii).
54. The use of item 52 or 53 further comprising taking into account information whether the subject suffers from non-small cell lung carcinoma subtype squamous cell carcinoma (SCC-NSCLC) or adenocarcinoma (ADC-NSCLC), preferably based on histological data.
55. The use of any one of items 52 to 53, further comprising the steps as defined in any one of items 1 to 50.
56. A computer-implemented method for assessing
   (i) the risk of NSCLC disease progression under an ongoing NSCLC treatment regime for a subject diagnosed for NSCLC;
   (ii) whether a subject diagnosed for NSCLC responds to an ongoing NSCLC treatment regime; and/or
   (iii) whether for a subject diagnosed for NSCLC an ongoing NSCLC treatment regime is to be maintained or modified;
   said computer implemented method comprising:
      a) receiving data comprising the level of CYFRA 21-1, CA 125 and/or CEA as determined in a) of any one of items 1 to 50 and optionally information on the NSCLC subtype; and
   b) processing said data to execute the comparing step as defined in b) of any one of items 1 to 50.
wherein said subject is classified to have a stable disease.
57. The method of item 56, wherein the method further comprises outputting the assessment (e.g. via a display).
58. A computer program product comprising instructions which, when the program is executed by a computer, cause the computer to carry out the method as defined in item 56 or 57.
59. A computer-readable medium comprising instructions which, when executed by a computer, cause the computer to carry out the method as defined in item 56 or 57.
60. A data processing system comprising a receiving unit configured to receiving data as defined in a) of item 56; and a processing unit configured to perform step b) of item 56; and optionally an outputting unit configured to output the assessment results.
61. A kit comprising:
   a reagent or a set of reagents for detecting the level of CYFRA 21-1 and/or a reagent or a set of reagents for detecting CA 125 in a sample obtained from a subject,
   wherein the kit is a kit for assessing:
   (i) the risk of NSCLC disease progression under an ongoing NSCLC treatment regime for a subject diagnosed for NSCLC;
   (ii) whether a subject diagnosed for NSCLC responds to an ongoing NSCLC treatment regime; and/or
   (iii) whether for a subject diagnosed for NSCLC an ongoing NSCLC treatment regime is to be maintained or modified;
   wherein said subject of (i), (ii) and (iii) is classified to have a stable disease.
62. The kit of item 61, further comprising a reagent or a set of reagents for detecting the level of CEA in a sample obtained from a subject.
63. The kit of item 61 or 62, wherein said kit comprises instructions how to provide the assessment as defined in (i), (ii) or (iii) of item 61, wherein said instructions include a description of the steps of a method as defined in any one of items 1 to 50 or the use as defined in any one of items 52 to 55 or refers to a computer implemented method of item 56 or 57, a computer program product of item 58, a computer-readable medium of item 59, or a data processing system of item 60.
64. The kit of any one of items 61 to 63, wherein the kit comprises a package insert stating one or more of the purposes as defined in (i) to (iii) of item 61.

### DETAILED DESCRIPTION OF THE INVENTION

It has been surprisingly found in the context of the present invention that determining protein levels of the biomarker CY FRA 21-1 and/or CA 125 in a sample (e.g. a serum or plasma sample), patients diagnosed to have a stable disease (SD) NSCLC (e,g, by imaging and RECIST criteria) can be further divided into two groups: a first group with a decreased risk for disease progression under the ongoing treatment regime and a second group with increased risk for disease progression under the ongoing treatment regime. By this risk stratification, also an assessment of the likelihood to respond to the ongoing treatment regime and/or aid in further treatment decisions, i.e. whether the ongoing treatment is to be maintained or modified, can be provided. A particularly surprising finding of the present invention was that the levels of CYFRA 21-1 and/or CA 125 at a single time point (especially at the time of the first CT after the second treatment cycle), e.g. as absolute concentrations or amounts (or a log transformation thereof), show a much better performance in disease progression risk assessment than using the changes in the levels of the same biomarkers relative to the treatment start.

As evident from the background section above, prior art studies by contrast focused on changes in biomarker levels to assess whether a patient responds to a treatment. Due to known fluctuation of the levels of the biomarkers used in the present invention between patients, it was absolutely unexpected that the risk stratification is superior when the level of the respective biomarker(s) before the treatment is not taken into account. Using a single biomarker level despite the better performance has also a number of other advantages. For example, it simplifies the biomarker based assessment since only a single biomarker measurement at a defined time point is required. Contrary to working with biomarker changes, the method of the invention can be conducted independent of the availability of prior biomarker levels and is thus available to an increased amount of patients. Moreover, the necessity of comparing biomarker levels from two independent time points and measurements that may potentially involve different assays is eliminated by the present invention.

With both biomarkers CYFRA 21-1 and CA 125 the univariate marker performance for assessing NSCLC progression risk (as evaluated e.g. by progression free survival and/or overall survival) was significantly better than for all other tumor biomarkers tested. For subjects suffering from a NSCLC of histological subtype adenocarcinoma, CYFRA 21-1 showed the best univariate performance. For subjects suffering from NSCLC of histological subtype squamous cell carcinoma (SCC-NSCLC), CA 125 showed the best univariate performance.

The combination of CYFRA 21-1 and CA 125 levels in form of a score further improved the performance. In particular, using an interaction term that takes into account the histological subtype (adenocarcinoma or SCC) for each of the biomarkers further improved the performance for calculating the score.

Further, it has been found that including a third biomarker level, namely the level of CEA, into the score can further improve the prognosis of disease progression under the ongoing treatment regime.

In a first aspect, the present invention relates to an in vitro method for assessing the risk of non-small cell lung carcinoma (NSCLC) disease progression for a subject.

The method according to the first aspect of the invention comprises:
a) determining the level of CYFRA 21-1 and/or the level of CA 125 in a sample obtained from the subject; and
b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score.

The subject from which the sample is obtained is a subject that is diagnosed to suffer from non-small cell lung carcinoma (NSCLC) and is under an ongoing NSCLC treatment regime. In other words, the subject from which the sample is obtained has been diagnosed before conducting the method according to the first aspect to suffer from NSCLC and is under an NSCLC treatment regime at the time point the sample was obtained.

Further, the subject from which the sample is obtained may be classified or diagnosed to have a stable disease by different means than the steps of the present method. Preferably, stable disease classification is done via an imaging method such as CT.

In embodiments, the subject from which the sample is obtained has been diagnosed to have a stable disease before or at the point in time the sample was obtained.

In embodiments, the sample is a sample that was obtained in a specific time frame around the day of the medical examination (e.g. imaging such as CT) leading to diagnosis of stable disease. In embodiments, the specific time frame may be from 35 days before the medical examination to 35 days after the medical examination. In a preferred embodiment the specific time frame may be from 30 days before the medical examination to 30 days after the medical examination. In an even more preferred embodiment, the specific time frame may be from 10 days before the medical examination to 29 days after the medical examination.

In embodiments, the subject is classified to have a stable disease based on data (e.g. tumor imaging data such as CT data) obtained at least 10 days, preferably at least 20 days and even more preferably at least 30 days, and most preferably at least 35 days after the start date of the ongoing treatment regime, in embodiments, the subject is classified to have a stable disease based on data (e.g. tumor imaging data such as CT data) obtained at most 150 days, preferably at most 120 days and most preferably at most 108 days after the start date of the ongoing treatment regime. Accordingly, the subject may be classified to have a stable disease based on data (e.g. tumor imaging data such as CT data) obtained 10 days to 150 days after the start date of the ongoing treatment regime, preferably 20 days to 120 days after the start date of the ongoing treatment regime and most preferably 3.5 days to 108 days after the start date of the ongoing treatment regime.

In embodiments, the sample is a sample obtained from the subject at least 10 days, preferably at least 20 days and most preferably at least 25 days after the start date of the ongoing treatment regime, in embodiments, the sample is a sample obtained from the subject at most 150 days, preferably at most 120 and most preferably at most 108 days after the start date of the ongoing treatment regime. Accordingly, in embodiments, the sample is a sample obtained from the subject 10 days to 150 days after the start date of the ongoing treatment regime, preferably 20 days to 120 days after the start date of the ongoing treatment regime, and most preferably 25 days to 108 days after the start date of the ongoing treatment regime.

In embodiments, the subject is classified to have a stable disease based on data (e.g. tumor imaging data) obtained 35 days to 108 days after the start date of the ongoing treatment regime and the sample is a sample obtained from the subject 25 days to 108 days after the start date of the ongoing treatment regime.

The “start” or “start date” of the ongoing treatment regime is the day the first treatment of this treatment regime was administered to the subject. For example, the day the drug or drug composition was administered the first time to the subject.

In embodiments of the method according to the first aspect, the level of CYFRA 21-1 may be determined in a) and the comparing in b) may comprises or consists of comparing the determined level of CYFRA 21-1 to a CYFRA 21-1 cut-off level, wherein a determined level of CYFRA 21-1 lower than or equal to the CYFRA 21-1 cut-off level is indicative for a low risk of NSCLC disease progression.

In embodiments of the method according to the first aspect, the level of CYFRA 21-1 is determined in a) and the comparing in b) comprises or consists of comparing the determined level of CYFRA 21-1 to a CYFRA 21-1 cut-off level, wherein a determined level of CYFRA 21-1 higher than the CYFRA 21-1 cut-off level is indicative for a high risk of NSCLC disease progression.

Accordingly, in embodiments of the first aspect of the invention provided is a method for assessing the risk of non-small cell lung carcinoma (NSCLC) disease progression for a subject comprising:
a) determining the level of CYFRA 21-1 in a sample obtained from the subject; and
b) comparing the determined level of CYFRA 21-1 to a CYFRA 21-1 cut-off level, wherein a determined level of CYFRA 21-1 lower than or equal to the CYFRA 21-1 cut-off level is indicative for a low risk of NSCLC disease progression, and/or wherein a determined level of CYFRA 21-1 higher than the CYFRA 21-1 cut-off level is indicative for a high risk of NSCLC disease progression,
wherein the subject from which the sample is obtained is a subject that is diagnosed to suffer from non-small cell lung carcinoma (NSCLC) and is under an ongoing NSCLC treatment regime, and wherein the subject is previously classified (or diagnosed) to have a stable disease stage. In this context, the subject is further preferably diagnosed for an NSCLC of subtype adenocarcinoma (ADC-NSCLC).

In the appended examples, it has been demonstrated that the performance of the biomarker CYFRA 21-1 in assessing the risk of non-small cell lung carcinoma (NSCLC) disease progression is particularly well for subjects that suffer from NSCLC of subtype adenocarcinoma. Thus, in embodiments determining the biomarker CYFRA 21-1, the subject from which the sample was obtained may in particular be a subject diagnosed to suffer from NSCLC of subtype adenocarcinoma.

In embodiments of the method according to the first aspect, the level of CA 125 may be determined in a) and the comparing in b) may comprise or consist of comparing the determined level of CA 125 to a CA 125 cut-off level, wherein a determined level of CA 125 lower than or equal to the CA 125 cut-off level is indicative for a low risk of NSCLC disease progression.

In embodiments of the method according to the first aspect, the level of CA 125 is determined in a) and the comparing in b) comprises or consists of comparing the determined level of CA 125 to a CA 125 cut-off level, wherein a determined level of CA 125 higher than the CA 125 cut-off level is indicative for a high risk of NSCLC disease progression.

Accordingly, in embodiments of the first aspect of the invention provided is a method for assessing the risk of non-small cell lung carcinoma (NSCLC) disease progression for a subject comprising:
a) determining the level of CA 125 in a sample obtained from the subject; and
b) comparing the determined level of CA 125 to a CA 125 cut-off level, wherein a determined level of CA 125 lower than or equal to the CA 125 cut-off level is indicative for a low risk of NSCLC disease progression, and/or wherein a determined level of CA 125 higher than the CA 125 cut-off level is indicative for a high risk of NSCLC disease progression,
   wherein the subject from which the sample is obtained is a subject that is diagnosed to suffer from non-small cell lung carcinoma (NSCLC) and is under an ongoing NSCLC treatment regime, and wherein the subject is previously classified (or diagnosed) to have a stable disease stage. In this context, the subject is further preferably diagnosed for an NSCLC of subtype squamous cell carcinoma (SCC-NSCLC).

In the appended examples, it has been demonstrated that the performance of the biomarker CA 125 in assessing the risk of non-small cell lung carcinoma (NSCLC) disease progression is particularly well for subjects that suffer from NSCLC of subtype squamous cell carcinoma (SCC). Thus, in embodiments determining the biomarker CA 125, the subject from which the sample was obtained, may be a subject diagnosed to suffer from NSCLC of subtype squamous cell carcinoma (SCC-NSCLC).

In embodiments of the first aspect of the present invention, the method may comprise determine a score taking into account the level of CYFRA 21-1 and/or CA 125 determined in the sample. In these embodiments, the comparison in b) comprises or consists of comparing the score taking into account the determined level of CYFRA 21-1 and/or the determined level of CA 125 to a cut-off score.

The score taking into account the level of CYFRA 21-1 and/or CA 125 in the sample may be determined by weighted calculation using the determined level(s) of CYFRA 21-1 and/or CA 125. In embodiments, the score may be based on CYFRA 21-1 and/or CA 125 only. In other embodiments, the score may take into account one or more other factors including but not limited to the presence or level of one or more other biomarkers in the sample and one or more clinical parameters of the subject (e.g. tumor histology, smoking status, stage of disease, age and/or sex). In a particular embodiment, the score may take into account whether the tumor histology is adenocarcinoma or SCC. Especially, the tumor histology may be taken into account by an interaction term with the biomarker values. Thereby, it can be taken into account that the biomarkers CYFRA 21-1 and CA 125 showed slightly different performance dependent on tumor histology.

Accordingly, the method according to the first aspect may be a method for assessing the risk of non-small cell lung carcinoma (NSCLC) disease progression for a subject, said method comprising:
a) determining the level of CYFRA 21-1 and/or the level of CA 125 in a sample obtained from the subject;
aa) determining a score taking into account the level of CYFRA 21-1 and/or the level of CA 125 determined in a); and
b) comparing the score taking into account the determined level of CYFRA 21-1 and/or the determined level of CA 125 to a cut-off score, wherein the subject from which the sample is obtained is a subject that is diagnosed to suffer from non-small cell lung carcinoma (NSCLC) and is under an ongoing NSCLC treatment regime, and wherein the subject is previously classified (or diagnosed) to have a stable disease stage.

In embodiments, a determined score (taking into account the determined level of CYFRA 21-1 and/or the determined level of CA 125) lower than the cut-off score is indicative for a low risk of NSCLC disease progression. In these embodiments, the score is configured such that the score is increased the higher the level of CYFRA 21-1 and/or the higher the level of CA 125 is.

In embodiments, a determined score (taking into account the determined level of CYFRA 21-1 and/or the determined level of CA 125) higher than the cut-off score is indicative for a high risk of NSCLC disease progression. In these embodiments., the score is configured such that the score is increased the higher the level of CYFRA 21-1 is and/or the higher the level of CA 125 is.

A skilled person will appreciate that a score taking into account the determined level of CYFRA 21-1 and/or the determined level of CA 125 can also be mathematically configured vice versa. e.g. that a score higher than the cut-off score is indicative for a lower risk and that a score higher than the cut-off score is indicative for a higher risk. This can be achieved by configuring the score such that the score decreases the higher the level of CYFRA 21-1 and/or the higher the level of CA 125 is. For instance, a reverse of the score can be achieved by using a negative factor multiplied with the biomarker level(s) in the formula for calculating the score.

In embodiments of the method of the first aspect, both the level of CYFRA 21-1 and the level of CA 125 in the sample are determined and the determined score takes into account both the determined level of CYFRA 21-1 and the determined level of CA 125.

Accordingly, the method according to the first aspect may be a method for assessing the risk of non-small cell lung carcinoma (NSCLC) disease progression for a subject, said method comprising:
a) determining the level of CYFRA 21-1 and the level of CA 125 in a sample obtained from the subject;
aa) determining a score taking into account the level of CYFRA 21-1 and the level of CA 125 determined in a); and
b) comparing the score taking into account the determined level of CYFRA 21-1 and the determined level of CA 125 to a cut-off score,
   wherein the subject from which the sample is obtained is a subject that is diagnosed to suffer from non-small cell lung carcinoma (NSCLC) and is under an ongoing NSCLC treatment regime, and wherein the subject is previously classified (or diagnosed) to have a stable disease stage.

The score taking into account the level of CYFRA 21-1 and CA 125 in the sample may be determined by weighted calculation using the determined level(s) of CYFRA 21-1 and/or CA 125.

In embodiments., the score taking into account the level of CY FRA 21-1 and CA 125 may be a binary score and the cut-off score may also be binary. “Binary” means that the score contains two values, e.g. a first value (also referred to as CYFRA 21-1 value) being the level of CYFRA 21-1 or a value derived therefrom and a second value (also referred to as CA125 value) being the level of CA125 or a value derived therefrom. A “value derived therefrom” can, for example, be a value obtained by a mathematical operation. The “value derived therefrom” is preferably direct proportional to the respective level. The values of the binary cut-off score may be obtained by univariate analysis with the respective biomarker, i.e. similar to the procedure of defining a cut-off for a single biomarker as described herein.

Comparing a binary score to a binary cut-off score means comparing the first value of the determined binary score to the first value of the cut-off binary score and the second value of the determined binary score to the second of the cut-off binary score. In embodiments, if both the first and the second value of the determined binary score are above the first and second value of the binary reference score, respectively, this is indicative for a high risk of NSCLC disease progression in the subject from which the sample was obtained. In embodiments, if one or both of the first and second value of the determined binary score are below the first and second value of the binary reference score, respectively, this is indicative for a low risk of NSCLC disease progression in the subject from which the sample was obtained.

In embodiments, a binary score in which the CYFRA 21-1 value of the determined binary score is above the CYFRA 21-1 cut-off value of the cut-off binary score and the CA 125 value of the determined binary score is above the CA 125 cut-off value of the cut-off binary score is indicative for a high risk of NSCLC disease progression in the subject from which the sample was obtained.

In embodiments, a binary score the CYFRA 21-1 value and/or the CA 125 value of the determined binary score is/fare below the CYFRA 21-1 cut-off value and/or the CA 125 cut-off value of the cut-off binary score is indicative for a low risk of NSCLC disease progression in the subject from which the sample was obtained.

Accordingly, the method according to the first aspect may be a method for assessing the risk of non-small cell lung carcinoma (NSCLC) disease progression for a subject, said method comprising:
a) determining the level of CYFRA 21-1 and the level of CA 125 in a sample obtained from the subject;
aa) determining a binary score comprising a first and a second value and taking into account the level of CYFRA 21-1 determined in a) in the first value and the level of CA 125 determined in a) in the second value;
b) comparing the binary score taking into account the determined level of CYFRA 21-1 and the determined level of CA 125 to a binary cut-off score, said binary cutoff score comprising a CYFRA 21-1 cut-off value and a CA 125 cut-off value, wherein the first value of the determined binary score is compared to the CYFRA 21-1 cut-off value and the second value of the determined binary score is compared to the CA 125 cut-off value;
c) assessing the risk of non-small cell lung carcinoma (NSCLC) disease progression in the subject based on the comparison in b),
   wherein the subject from which the sample is obtained is a subject that is diagnosed to suffer from non-small cell lung carcinoma (NSCLC) and is under an ongoing NSCLC treatment regime, and wherein the subject is previously classified (or diagnosed) to have a stable disease stage.

In embodiments of the method according to the first aspect, the risk of NSCLC disease progression is assessed relative to the mean or median NSCLC disease progression risk of a reference population, such as the reference population on which the respective cut-off or cut-off score is based. The reference population may preferably be a population of subjects suffering from non-small cell lung carcinoma (NSCLC) and being categorized/diagnosed as stable disease based on imaging (e.g. using CT. As demonstrated in the appended Examples detecting the level of CYFRA 21-1 and/or the level of CA 125 or a score taking into account the level(s) provides a further risk stratification within these group of subjects and allows to separate this patient population into patients having a lower disease progression risk comparable to subjects being categorized to have a partial response (PR) and patients having a higher disease progression risk comparable to subjects having progressive disease (PD).

In embodiments, high disease progression risk may be a disease progression risk comparable to the average disease progression risk of reference population that consists of subjects being categorized as having progressive NSCLC disease according to imaging (e.g. CT based imaging).

In embodiments, low disease progression risk may be a disease progression risk comparable to the average disease progression risk of reference population that consists of subjects being categorized as having partial response to the NSCLC treatment according to imaging (e.g. CT based imaging).

In embodiments, high disease progression risk may be at least 1.5 fold, preferably at least 2-fold higher than low disease progression risk. In other words, the ratio between the high and the low progression risk may amount to at least 1.5, preferably at least 2. In embodiments, the ratio may be a hazard ratio.

In embodiments, the risk of NSCLC disease progression includes the risk for tumor growth (preferably as assessed by RECIST criteria), the risk for formation of new lesions or metastasis or and/or the risk of NSCLC caused death.

The method according to the first aspect may further comprise determining the protein level of CEA in the sample. In these embodiments, the comparing step comprises comparing a score taking into account the determined level of CYFRA 21-1 and/or the determined level of CA 125 and the determined level of CEA to a cut-off score.

Accordingly, herein provided is an in vitro method for assessing the risk of non-small cell lung carcinoma (NSCLC) disease progression for a subject suffering from non-small cell lung carcinoma (NSCLC) and being under an ongoing NSCLC treatment regime, said method comprising:
a) determining the level of CYFRA 21-1, the level of CA 125 and the level of CEA in a sample obtained from the subject;
aa) determining a score taking into account the level of CYFRA 21-1, the level of CA 125 and the level of CEA in the sample; and
b) comparing the score determined in aa) to a cut-off score,
   wherein the subject is previously classified to have a stable disease (e.g. by imaging such as CT).

In embodiments, the method according to the first aspect may further comprise obtaining the information whether the subject suffers from non-small cell lung carcinoma of subtype squamous cell carcinoma (SCC-NSCLC) or of subtype adenocarcinoma (ADC-NSCLC). Preferably, the obtained information is based on histological data.

As demonstrated in the appended examples, when using scores taking into account at least CYFRA 21-1 and CA 125 levels (and optionally CEA), further including information regarding the histological subtype of the NSCLC (adenocarcinoma or SCC), e.g. by including interaction terms between the determined biomarkers and the histology type in the mathematical formula for calculating the score, the performance in assessing disease progression risk of SD patients was further increased.

In embodiments, the method of the first aspect comprises obtaining information about the NSCLC histological subtype (adenocarcinoma or SCC) and the comparing step comprises comparing a score taking into account the level of the determined biomarker (i.e. CYFRA 21-1 and/or CA 125 and optionally CEA) and the histological NSCLC subtype. In embodiments, the score takes the histological subtype into account by one or more interaction terms between the histological NSCLC subtype and one or more of the determined biomarkers.

Accordingly, in one embodiment of the first aspect, provided is an in vitro method for assessing the risk of non-small cell lung carcinoma (NSCLC) disease progression for a subject suffering from non-small cell lung carcinoma (NSCLC) and being under an ongoing NSCLC treatment regime, said method comprising:
a) determining the level of CYFRA 21-1 and the level of CA 125 in a sample obtained from the subject;
aa) obtaining information regarding the histological NSCLC subtype from which the subject suffers
aaa) determining a score taking into account: i) the level of CYFRA 21-1 and the level of CA 125 in the sample; and ii) the histological NSCLC subtype from which the subject suffers (e.g. in form of interaction terms between the biomarker levels and the histological subtype); and
b) comparing the score determined in aaa) to a cut-off score,
   wherein the subject is previously classified to have a stable disease (e.g. by imaging such as CT).

In another embodiment of the first aspect, provided is a an in vitro method for assessing the risk of non-small cell lung carcinoma (NSCLC) disease progression for a subject suffering from non-small cell lung carcinoma (NSCLC) and being under an ongoing NSCLC treatment regime, said method comprising:
a) determining the level of CYFRA 21-1, the level of CA 125 and the level of CEA in a sample obtained from the subject;
aa) obtaining information regarding the histological NSCLC subtype from which the subject suffers
aaa) determining a score taking into account: i) the level of CYFRA 21-1, the level of CA 125 and the level of CEA in the sample; and ii) the histological NSCLC subtype from which the subject suffers (e.g. in form of interaction terms between the biomarker levels and the histological subtype); and
b) comparing the score determined in aaa) to a cut-off score, wherein the subject is previously classified to have a stable disease (e.g. by imaging such as CT).

In embodiments of the method according to the first aspect, the method may further comprise providing the information whether the subject is at high or low risk for disease progression to the subject and/or a physician. This information may then be used to aid further treatment decisions in that it provides a valuable additional input for treatment response that goes beyond mere imaging results.

In a second aspect, the present invention relates to an in vitro method for assessing whether for a subject diagnosed for non-small cell lung carcinoma (NSCLC) an ongoing NSCLC treatment regime is to be maintained or modified, said method comprising:
a) determining the level of CYFRA 21-1 and/or the level of CA 125 in a sample obtained from the subject; and
b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score;
   wherein the subject is a subject that is classified or diagnosed to have a stable disease (e.g. by tumor imaging based on CT).

Accordingly, the subject from which the sample was obtained is a subject that is diagnosed to suffer from non-small cell lung carcinoma (NSCLC) and is under an ongoing NSCLC treatment regime. In other words, the subject from which the sample is obtained has been diagnosed before conducting the method according to the second aspect to suffer from NSCLC and is under an NSCLC treatment regime at the time point the sample was obtained.

Further, the subject from which the sample is obtained may be classified or diagnosed to have a stable disease by different means than the steps of the present method. Preferably, stable disease classification is done via an imaging method such as CT.

In embodiments, the subject from which the sample is obtained has been diagnosed to have a stable disease before or at the point in time the sample was obtained.

In embodiments, the sample is a sample that was obtained in a specific time frame around the day of the medical examination (e.g. imaging such as CT) leading to diagnosis of stable disease. In embodiments, the specific time frame may be from 35 days before the medical examination to 35 days after the medical examination. In a preferred embodiment the specific time frame may be from 30 days before the medical examination to 30 days after the medical examination. In an even more preferred embodiment, the specific time frame may be from 10 days before the medical examination to 29 days after the medical examination.

In embodiments, the subject is classified to have a stable disease based on data (e.g. tumor imaging data such as CT data) obtained at least 10 days, preferably at least 20 days and even more preferably at least 30 days, and most preferably at least 35 days after the start date of the ongoing treatment regime. In embodiments, the subject is classified to have a stable disease based on data (e.g. tumor imaging data such as CT data) obtained at most 150 days, preferably at most 120 days and most preferably at most 108 days after the start date of the ongoing treatment regime. Accordingly, the subject may be classified to have a stable disease based on data (e.g. tumor imaging data such as CT data) obtained 10 days to 150 days after the start date of the ongoing treatment regime, preferably 20 days to 120 days after the start date of the ongoing treatment regime and most preferably 35 days to 108 days after the start date of the ongoing treatment regime.

In embodiments, the sample is a sample obtained from the subject at least 10 days, preferably at least 20 days and most preferably at least 25 days after the start date of the ongoing treatment regime. In embodiments, the sample is a sample obtained from the subject at most ISO days, preferably at most 120 and most preferably at most 108 days after the start date of the ongoing treatment regime. Accordingly, in embodiments, the sample is a sample obtained from the subject 10 days to 150 days after the start date of the ongoing treatment regime, preferably 20 days to 120 days after the start date of the ongoing treatment regime, and most preferably 25 days to 108 days after the start date of the ongoing treatment regime.

In embodiments, the subject is classified to have a stable disease based on data (e.g. tumor imaging data) obtained 35 days to 108 days after the start date of the ongoing treatment regime and the sample is a sample obtained from the subject 25 days to 108 days after the start date of the ongoing treatment regime.

The “start” or “start date” of the ongoing treatment regime is the day the first treatment of this treatment regime was administered to the subject. For example, the day the drug or drug composition was administered the first time to the subject.

In embodiments of die method according to the second aspect, the level of CYFRA 21-1 may be determined in a) and the comparing in b) may comprises or consists of comparing the determined level of CYFRA 21-1 to a CYFRA 21-1 cut-off level, wherein a determined level of CYFRA 21-1 lower than or equal to the CYFRA 21-1 cut-off level indicates that the ongoing NSCLC treatment regime is to be maintained.

In embodiments of the method according to the second aspect, the level of CYFRA 21-1 is determined in a) and the comparing in b) comprises or consists of comparing the determined level of CYFRA 21-1 to a CYFRA 21-1 cut-off level, wherein a determined level of CYFRA 21-1 higher than the CYFRA 21-1 cut-off level indicates that the ongoing NSCLC treatment regime is to be modified.

Accordingly, in embodiments of the second aspect of the invention provided is a method for assessing whether for a subject diagnosed for non-small cell lung carcinoma (NSCLC) an ongoing NSCLC treatment regime is to be maintained or modified, said method comprising:
a) determining the level of CYFRA 21-1 in a sample obtained from the subject; and
b) comparing the determined level of CYFRA 21-1 to a CYFRA 21-1 cut-off level, wherein a determined level of CYFRA 21-1 lower than or equal to the CYFRA 21-1 cut-off level indicates that the ongoing NSCLC treatment regime is to be maintained, and/or wherein a determined level of CYFRA 21-1 higher than the CYFRA 21-1 cut-off level indicates that the ongoing NSCLC treatment regime is to be modified, wherein the subject is previously classified (or diagnosed) to have a stable disease stage (e.g. by CT based imaging). In this context, the subject is further preferably diagnosed for an NSCLC of subtype adenocarcinoma (ADC-NSCLC).

In the appended examples, it has been demonstrated that the performance of the biomarker CYFRA 21-1 in assessing the risk of non-small cell lung carcinoma (NSCLC) disease progression and consequently treatment response is particularly well for subjects that suffer from NSCLC of subtype adenocarcinoma. Thus, in embodiments determining the biomarker CYFRA 21-1, the subject, from which the sample is obtained, may especially be a subject previously diagnosed to suffer from NSCLC of subtype adenocarcinoma.

In embodiments of the method according to the second aspect, the level of CA 125 may be determined in a) and the comparing in b) may comprise or consist of comparing the determined level of CA 125 to a CA 125 cut-off level, wherein a determined level of CA 125 lower than or equal to the CA 125 cut-off level indicates that the ongoing NSCLC treatment regime is to be maintained.

In embodiments of the method according to the second aspect, the level of CA 125 is determined in a) and the comparing in b) comprises or consists of comparing the determined level of CA 125 to a CA 125 cut-off level, wherein a determined level of CA 125 higher than the CA 125 cut-off level indicates that the ongoing NSCLC treatment regime is to be modified.

Accordingly, in embodiments of the second aspect of the invention provided is a method for assessing whether for a subject diagnosed for non-small cell lung carcinoma (NSCLC) an ongoing NSCLC treatment regime is to be maintained or modified, said method comprising:
a) determining the level of CA 125 in a sample obtained from the subject; and
b) comparing the determined level of CA 125 to a CA 125 cut-off level, wherein a determined level of CA 125 lower than or equal to the CA 125 cut-off level indicates that the ongoing NSCLC treatment regime is to be maintained, and/or wherein a determined level of CA 125 higher than the CA 125 cut-off level indicates that the ongoing NSCLC treatment regime is to be modified, wherein the subject is previously classified (or diagnosed) to have a stable disease stage. In this context, the subject is further preferably diagnosed for an NSCLC of subtype squamous cell carcinoma (SCC-NSCLC).

In the appended examples is has been demonstrated that the performance of the biomarker CA 125 in assessing the risk of non-small cell lung carcinoma (NSCLC) disease progression is particularly good for subjects that suffer from NSCLC of subtype squamous cell carcinoma. Thus, in embodiments determining the biomarker CA 125, the subjects from which the sample may in particular by a subject diagnosed to suffer from NSCLC of subtype squamous cell carcinoma (SCC-NSCLC).

In embodiments of the second aspect of the present invention, the method may comprise determine a score taking into account the level of CYFRA 21-1 and/or CA 125 determined in the sample. In these embodiments, the comparison in b) comprises or consists of comparing the score taking into account the determined level of CYFRA 21-1 and/or the determined level of CA 125 to a cut-off score.

The score taking into account the level of CYFRA 21-1 and/or CA 125 in the sample may be determined by weighted calculation using the determined level(s) of CYFRA 21-1 and/or CA 125. In embodiments, the score may be based on CYFRA 21-1 and/or CA 125 only. In other embodiments, the score may take into account one or more other factors including but not limited to the presence or level of one or more other biomarkers in the sample and one or more clinical parameters of the subject (e.g. tumor histology, smoking status, stage of disease, age and/or sex). In a particular embodiment, the score may take into account whether the tumor histology is adenocarcinoma or SCC. Especially, the tumor histology may be taken into account by an interaction term with the biomarker values. Thereby, it can be taken into account that the biomarkers CYFRA 21-1 and CA 125 showed slightly different performance dependent on tumor histology.

Accordingly, the method according to the second aspect may be a method for assessing whether for a subject diagnosed for non-small cell lung carcinoma (NSCLC) an ongoing NSCLC treatment regime is to be maintained or modified, said method comprising:
a) determining the level of CYFRA 21-1 and/or the level of CA 125 in a sample obtained from the subject;
aa) determining a score taking into account the level of CYFRA 21-1 and/or the level of CA 125 determined in a); and
b) comparing the score taking into account the determined level of CYFRA 21-1 and/or the determined level of CA 125 to a cut-off score,
   wherein the subject has been previously classified (or diagnosed) to have a stable disease stage.

In embodiments, a determined score (taking into account the determined level of CYFRA 21-1 and/or the determined level of CA 125) lower than the cut-off score indicates that the ongoing NSCLC treatment regime is to be maintained. In these embodiments, the score is configured such that the score is increased the higher the level of CYFRA 21-1 and/or the higher the level of CA 125 is.

In embodiments, a determined score (taking into account the determined level of CYFRA 21-1 and/or the determined level of CA 125) higher than the cut-off score indicates that the ongoing NSCLC treatment regime is to be modified. In these embodiments, the score is configured such that the score is increased the higher the level of CYFRA 21-1 is and/or the higher the level of CA 125 is.

A skilled person will appreciate that a score taking into account the determined level of CYFRA 21-1 and/or the determined level of CA 125 can also be mathematically configured vice versa, e.g. that a score higher than the cut-off score is indicative for a lower risk and that a score higher than the cut-off score is indicative for a higher risk. This can be achieved by configuring the score such that the score decreases the higher the level of CYFRA 21-1 and/or the higher the level of CA 125 is. For instance, a reverse of the score can be achieved by using a negative factor multiplied with the biomarker level(s) in the formula for calculating the score.

In embodiments of the method of the second aspect, both the level of CYFRA 21-1 and the level of CA 125 in the sample are determined and the determined score takes into account both the determined level of CYFRA 21-1 and the determined level of CA 125.

Accordingly, the method according to the second aspect may be a method for assessing whether for a subject diagnosed for non-small cell lung carcinoma (NSCLC) an ongoing NSCLC treatment regime is to be maintained or modified, said method comprising:
a) determining the level of CYFRA 21-1 and the level of CA 125 in a sample obtained from the subject;
aa) determining a score taking into account the level of CYFRA 21-1 and the level of CA 125 determined in a); and
b) comparing the score taking into account the determined level of CYFRA 21-1 and the determined level of CA 125 to a cut-off score,
   wherein the subject is previously classified (or diagnosed) to have a stable disease stage.

The score taking into account the level of CYFRA 21-1 and CA 125 in the sample may be determined by weighted calculation using the determined level(s) of CYFRA 21-1 and/or CA 125.

In embodiments, the score taking into account the level of CYFRA 21-1 and CA 125 may be a binary score and the cut-off score may also be binary. “Binary” means that the score contains two values, e.g. a first value (also referred to as CYFRA 21-1 value) being the level of CYFRA 21-1 or a value derived therefrom and a second value (also referred to as CA125 value) being the level of CA125 or a value derived therefrom. A “value derived therefrom” can, for example, be a value obtained by a mathematical operation. The “value derived therefrom” is preferably direct proportional to the respective level. The values of the binary cut-off score may be obtained by univariate analysis with the respective biomarker, i.e, similar to the procedure of defining a cut-off for a single biomarker as described herein.

Comparing a binary score to a binary cut-off score contains comparing the first value of the determined binary score to the first value of the cut-off binary score and the second value of the determined binary score to the second of the cut-off binary score. In embodiments, if both the first and the second value of the determined binary score are above the first and second value of the binary reference score, respectively, this indicates that the ongoing NSCLC treatment regime is to be modified. In embodiments, if one or both of the first and second value of the determined binary score are below the first and second value of the binary reference score, respectively, this indicates that the ongoing NSCLC treatment regime is to be maintained.

In embodiments, a binary score in which the CYFRA 21-1 value of the determined binary score is above the CYFRA 21-1 cut-off value of the cut-off binary score and the CA 125 value of the determined binary score is above the CA 125 cut-off value of the cut-off binary score indicates that the ongoing NSCLC treatment regime is to be modified.

In embodiments, a binary score the CYFRA 21-1 value and/or the CA 125 value of the determined binary score is/are below the CYFRA 21-1 cut-off value and/or the CA 125 cut-off value of the cut-off binary score indicates that the ongoing NSCLC treatment regime is to be maintained.

Accordingly, the method according to the second aspect may be a method for assessing whether for a subject diagnosed for non-small cell lung carcinoma (NSCLC) an ongoing NSCLC treatment regime is to be maintained or modified, said method comprising:
a) determining the level of CYFRA 21-1 and the level of CA 125 in a sample obtained from the subject;
aa) determining a binary score comprising a first and a second value and taking into account the level of CYFRA 21-1 determined in a) in the first value and the level of CA 125 determined in a) in the second value;
b) comparing the binary score taking into account the determined level of CYFRA 21-1 and the determined level of CA 125 to a binary cut-off score, said binary cutoff score comprising a CYFRA 21-1 cut-off value and a CA 125 cut-off value, wherein the first value of the determined binary score is compared to the CYFRA 21-1 cut-off value and the second value of the determined binary score is compared to the CA 125 cut-off value;
c) assessing whether the ongoing NSCLC treatment regime is to be maintained or modified based on the comparison in b),
   wherein the subject from which the sample is obtained is a subject that is diagnosed to suffer from non-small cell lung carcinoma (NSCLC) and is under an ongoing NSCLC treatment regime, and wherein the subject is previously classified (or diagnosed) to have a stable disease stage.

The method according to the second aspect may further comprise determining the level of CEA in the sample. In these embodiments, the comparing step comprises comparing a score taking into account the determined level of CYFRA 21-1 and/or the determined level of CA 125 and the determined level of CEA to a cut-off score. Accordingly, in one embodiment of the second aspect of the invention provided is an in vitro method for assessing whether for a subject diagnosed for non-small cell lung carcinoma (NSCLC) an ongoing NSCLC treatment regime is to be maintained or modified, said method comprising:
a) determining the level of CYFRA 21-1, the level of CA 125 and the level of CEA in a sample obtained from the subject;
aa) determining a score taking into account the level of CYFRA 21-1, the level of CA 125 and the level of CEA in the sample; and
b) comparing the score determined in aa) the to a cut-off score,
   wherein the subject is previously classified to have a stable disease (e.g. by imaging such as CT).

In embodiments, the method according to the second aspect may further comprise obtaining the information whether the subject suffers from non-small cell lung carcinoma of subtype squamous cell carcinoma (SCC-NSCLC) or of subtype adenocarcinoma (ADC-NSCLC). Preferably, the obtained information is based on histological data.

As demonstrated in the appended examples using scores taking into account CYFRA 21-1 and CA 125 levels (and optionally CEA), the further inclusion of information regarding the histological subtype of the NSCLC (adenocarcinoma or SCC) into the score, e.g. by including interaction terms between the determined biomarkers and the histology type in the mathematical formula for calculating the score, the performance in assessing disease progression risk of SD patients was further increased.

In embodiments, the method of the second aspect comprises obtaining information about the NSCLC histological subtype (adenocarcinoma or SCC) and the comparing step comprises comparing a score taking into account the level of the determined biomarker (i.e. CYFRA 21-1 and/or CA 125 and optionally CEA) and the histological NSCLC subtype. In embodiments, the score takes the histological subtype into account by one or more interaction terms between the histological NSCLC subtype and one or more of the determined biomarkers.

Accordingly, in one embodiment of the second aspect, provided is an in vitro method for assessing whether for a subject diagnosed for non-small cell lung carcinoma (NSCLC) an ongoing NSCLC treatment regime is to be maintained or modified, said method comprising:
a) determining the level of CYFRA 21-1 and the level of CA 125 in a sample obtained from the subject;
aa) obtaining information regarding the histological NSCLC subtype from which the subject suffers;
aaa) determining a score taking into account: i) the level of CYFRA 21-1 and the level of CA 125 in the sample; and ii) the histological NSCLC subtype from which the subject suffers (e.g. in form of interaction terms between the biomarker levels and the histological subtype); and
b) comparing the score determined in aaa) the to a cut-off score,
   wherein the subject is previously classified to have a stable disease (e.g. by imaging such as CT).

In another embodiment of the second aspect, provided is a an in vitro method for assessing whether for a subject diagnosed for non-small cell lung carcinoma (NSCLC) an ongoing NSCLC treatment regime is to be maintained or modified, said method comprising:
a) determining the level of CYFRA 21-1, the level of CA 125 and the level of CEA in a sample obtained from the subject;
aa) obtaining information regarding the histological NSCLC subtype from which the subject suffers;
aaa) determining a score taking into account: i) the level of CYFRA 21-1, the level of CA 125 and the level of CEA in the sample; and ii) the histological NSCLC subtype from which the subject suffers (e.g. in form of interaction terms between the biomarker levels and the histological subtype); and
b) comparing the score determined in aaa) the to a cut-off score,
   wherein the subject is previously classified to have a stable disease (e.g. by imaging such as CT).

In embodiments of the method according to the second aspect, the method may further comprise providing the information obtained by the method. i.e. the information whether the treatment is to be maintained or modified to the subject and/or a physician. This information may then be used to aid further treatment decisions in that it provides a valuable additional input for treatment response that goes beyond mere imaging results.

In embodiments of the method according to the second aspect, “maintaining the treatment regime” means that the ongoing treatment regime is continued, i.e. that further treatment cycles take place.

In embodiments of the method according to the second aspect, “modifying the treatment regime” includes but is not limited to: (i) adjusting the dose of the treatment (e.g. increasing the dose of the drug), (ii) changing the type of treatment to another type of treatment (e.g. if the ongoing treatment is a chemotherapy, modifying the treatment may be changing the treatment to an immunotherapy) In other words “modifying the ongoing treatment regime” may be expressed as “adjusting the ongoing treatment regime” or “changing the ongoing treatment regime”.

In a third aspect, the present invention relates to an in vitro method for assessing whether a subject suffering from non-small cell lung carcinoma (NSCLC) responds to an ongoing NSCLC treatment regime, said method comprising:
a) determining die level of CYFRA 21-1 and/or the level of CA 125 in a sample obtained from the subject; and
b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score.

The subject in the context of this method is a subject classified or diagnosed to have a stable disease.

Accordingly, the subject from which the sample was obtained is a subject that is diagnosed to suffer from non-small cell lung carcinoma (NSCLC) and is under an ongoing NSCLC treatment regime. In other words, the subject from which the sample is obtained has been diagnosed before conducting the method according to the third aspect to suffer from NSCLC and is under an NSCLC treatment regime at the time point the sample was obtained.

Further, the subject from which the sample is obtained may be classified or diagnosed to have a stable disease by different means than the steps of the present method. Preferably, stable disease classification is done via an imaging method such as CT.

In embodiments, the subject from which the sample is obtained has been diagnosed to have a stable disease before or at the point in time the sample was obtained.

In embodiments, the sample is a sample that was obtained in a specific time frame around the day of the medical examination (e.g. imaging such as CT) leading to diagnosis of stable disease. In embodiments, the specific time frame may be from 35 days before the medical examination to 35 days after the medical examination. In a preferred embodiment the specific time frame may be from 30 days before the medical examination to 30 days after the medical examination. In an even more preferred embodiment, the specific time frame may be from 10 days before the medical examination to 29 days after the medical examination.

In embodiments, the subject is classified to have a stable disease based on data (e.g. tumor imaging data such as CT data) obtained at least 10 days, preferably at least 20 days and even more preferably at least 30 days, and most preferably at least 35 days after the start date of the ongoing treatment regime. In embodiments, the subject is classified to have a stable disease based on data (e.g. tumor imaging data such as CT data) obtained at most 150 days, preferably at most 120 days and most preferably at most 108 days after the start date of the ongoing treatment regime. Accordingly, the subject may be classified to have a stable disease based on data (e.g. tumor imaging data such as CT data) obtained 10 days to 150 days after the start date of the ongoing treatment regime, preferably 20 days to 120 days after the start date of the ongoing treatment regime and most preferably 35 days to 108 days after the start date of the ongoing treatment regime.

In embodiments, the sample is a sample obtained from the subject at least 10 days, preferably at least 20 days and most preferably at least 25 days after the start date of the ongoing treatment regime. In embodiments, the sample is a sample obtained from the subject at most ISO days, preferably at most 120 and most preferably at most 108 days after the start date of the ongoing treatment regime. Accordingly, in embodiments, the sample is a sample obtained from the subject 10 days to 150 days after the start date of the ongoing treatment regime, preferably 20 days to 120 days after the start date of the ongoing treatment regime, and most preferably 25 days to 108 days after the start date of the ongoing treatment regime.

In embodiments, the subject is classified to have a stable disease based on data (e.g. tumor imaging data) obtained 35 days to 108 days after the start date of the ongoing treatment regime and the sample is a sample obtained from the subject 25 days to 108 days after the start date of the ongoing treatment regime.

The “start” or “start date” of the ongoing treatment regime is the day the first treatment of this treatment regime was administered to the subject. For example, the day the drug or drug composition was administered the first time to the subject.

In embodiments of the third aspect of the invention, determining indicating whether the subject responds to the ongoing treatment may be determining/indicating whether the subject has responded to the ongoing treatment regime at the time at which the sample was obtained.

In embodiments of the third aspect of the invention, determining/indicating whether the subject responds to the ongoing treatment may be determining/indicating whether the subject will respond (i.e. responds after the sample was obtained) to the ongoing treatment regime.

In embodiments of the method according to the third aspect, the level of CYFRA 21-1 may be determined in a) and the comparing in b) may comprises or consists of comparing the determined level of CYFRA 21-1 to a CYFRA 21-1 cut-off level, wherein a determined level of CYFRA 21-1 lower than or equal to the CYFRA 21-1 cut-off level indicates that the subject responds to the ongoing NSCLC treatment regime.

In embodiments of the method according to the third aspect, the level of CY FRA 21-1 is determined in a) and the comparing in b) comprises or consists of comparing the determined level of CYFRA 21-1 to a CYFRA 21-1 cut-off level, wherein a determined level of CYFRA 21-1 higher than the CYFRA 21-1 cut-off level indicates that the subject does not respond to the ongoing NSCLC treatment regime.

Accordingly, in embodiments of the third aspect of the invention provided is an in vitro method for assessing whether a subject suffering from non-small cell lung carcinoma (NSCLC) responds to an ongoing NSCLC treatment regime, said method comprising:
a) determining the level of CYFRA 21-1 in a sample obtained from the subject; and
b) comparing the determined level of CYFRA 21-1 to a CYFRA 21-1 cut-off level, wherein a determined level of CYFRA 21-4 lower than or equal to the CYFRA 21-1 cut-off level indicates that the subject responds to the ongoing NSCLC treatment regime, and/or wherein a determined level of CYFRA 21-1 higher than the CYFRA 21-1 cut-off level indicates that the subject does not respond to the ongoing NSCLC treatment regime. In this context, the subject is further preferably diagnosed for an NSCLC of adenocarcinoma (ADC-NSCLC).
   wherein the subject is previously classified (or diagnosed) to have a stable disease stage (e.g. by CT based imaging). In this context, the subject is further preferably diagnosed for an NSCLC of subtype squamous cell carcinoma (ADC-NSCLC).

In the appended examples, it has been demonstrated that the performance of the biomarker CYFRA 21-1 in assessing the risk of non-small cell lung carcinoma (NSCLC) disease progression and consequently also detection and prognosis of treatment response is particularly well for subjects that suffer from NSCLC of subtype adenocarcinoma. Thus, in embodiments determining the biomarker CYFRA 21-1, the subject, from which the sample is obtained, may especially be a subject previously diagnosed to suffer from NSCLC of subtype adenocarcinoma.

In embodiments of the method according to the third aspect, the level of CA 125 may be determined in a) and the comparing in b) may comprise or consist of comparing the determined level of CA 125 to a CA 125 cut-off level, wherein a determined level of CA 125 lower than or equal to the CA 125 cut-off level indicates that the subject responds to the ongoing NSCLC treatment regime.

In embodiments of the method according to the third aspect, the level of CA 125 is determined in a) and the comparing in b) comprises or consists of comparing the determined level of CA 125 to a CA 125 cut-off level, wherein a determined level of CA 125 higher than the CA 125 cut-off level indicates that the subject does not respond to the ongoing NSCLC treatment regime.

Accordingly, in embodiments of the third aspect of the invention provided is an in vitro method for assessing whether a subject suffering from non-small cell lung carcinoma (NSCLC) responds to an ongoing NSCLC treatment regime, said method comprising:
a) determining the level of CA 125 in a sample obtained from the subject; and
b) comparing the determined level of CA 125 to a CA 125 cut-off level, wherein a determined level of CA 125 lower than or equal to the CA 125 cut-off level indicates that the ongoing NSCLC treatment regime is to be maintained, and/or wherein a determined level of CA 125 higher than the CA 125 cut-off level indicates that the ongoing NSCLC treatment regime is to be modified, wherein the subject is previously classified (or diagnosed) to have a stable disease stage. In this context, the subject is further preferably diagnosed for an NSCLC of subtype squamous cell carcinoma (SCC-NSCLC).

In the appended examples is has been demonstrated that the performance of the biomarker CA 125 in assessing the risk of non-small cell lung carcinoma (NSCLC) disease progression is particularly good for subjects that suffer from NSCLC of subtype squamous cell carcinoma. Thus, in embodiments determining the biomarker CA 125, the subjects from which the sample may in particular by a subject diagnosed to suffer from NSCLC of subtype squamous cell carcinoma (SCC-NSCLC).

In embodiments of the third aspect of the present invention, the method may comprise determine a score taking into account the level of CYFRA 21-1 and/or CA 125 determined in the sample. In these embodiments, the comparison in b) comprises or consists of comparing the score taking into account the determined level of CYFRA 21-1 and/or the determined level of CA 125 to a cut-off score.

The score taking into account the level of CYFRA 21-1 and/or CA 125 in the sample may be determined by weighted calculation using the determined level(s) of CYFRA 21-1 and/or CA 125. In embodiments, the score may be based on CYFRA 21-1 and/or CA 125 only. In other embodiments, the score may take into account one or more other factors including but not limited to the presence or level of one or more other biomarkers in the sample and one or more clinical parameters of the subject (e.g. tumor histology, smoking status, stage of disease, age and/or sex). In a particular embodiment, the score may take into account whether the tumor histology is adenocarcinoma or SCC. Especially, the tumor histology may be taken into account by an interaction term with the biomarker values. Thereby, it can be taken into account that the biomarkers CYFRA 21-1 and CA 125 showed slightly different performance dependent on tumor histology.

Accordingly, the method according to the third aspect may be a method for assessing whether a subject suffering from non-small cell lung carcinoma (NSCLC) responds to an ongoing NSCLC treatment regime, said method comprising:
a) determining the level of CYFRA 21-1 and/or the level of CA 125 in a sample obtained from the subject;
aa) determining a score taking into account the level of CYFRA 21-1 and/or the level of CA 125 determined in a); and
b) comparing the score taking into account the determined level of CYFRA 21-1 and/or the determined level of CA 125 to a cut-off score,
   wherein the subject has been previously classified (or diagnosed) to have a stable disease stage.

In embodiments, a determined score (taking into account the determined level of CYFRA 21-1 and/or the determined level of CA 125) lower than the cut-off score indicates that the subject responds to the ongoing NSCLC treatment regime. In these embodiments, the score is configured such that the score is increased the higher the level of CYFRA 21-1 and/or the higher the level of CA 125 is.

In embodiments, a determined score (taking into account the determined level of CYFRA 21-1 and/or the determined level of CA 125) higher than the cut-off score indicates that the subject does not respond to the ongoing NSCLC treatment regime. In these embodiments, the score is configured such that the score is increased the higher the level of CYFRA 21-1 is and/or the higher the level of CA 125 is.

A skilled person will appreciate that a score taking into account the determined level of CYFRA 21-1 and/or the determined level of CA 125 can also be mathematically configured vice versa, e.g. that a score higher than the cut-off score is indicative for a lower risk and that a score higher than the cut-off score is indicative for a higher risk. This can be achieved by configuring the score such that the score decreases the higher the level of CYFRA 21-1 and/or the higher the level of CA 125 is. For instance, a reverse of the score can be achieved by using a negative factor multiplied with the biomarker level(s) in the formula for calculating the score.

In embodiments of the method of the third aspect, both the level of CYFRA 21-1 and the level of CA 125 in the sample are determined and the determined score takes into account both the determined level of CYFRA 21-1 and the determined level of CA 125.

Accordingly, the method according to the third aspect may be a method for assessing whether a subject suffering from non-small cell lung carcinoma (NSCLC) responds to an ongoing NSCLC treatment regime, said method comprising:
a) determining the level of CYFRA 21-1 and the level of CA 125 in a sample obtained from the subject;
aa) determining a score taking into account the level of CYFRA 21-1 and the level of CA 125 determined in a); and
b) comparing the score taking into account the determined level of CYFRA 21-1 and the determined level of CA 125 to a cut-off score,
   wherein the subject is previously classified (or diagnosed) to have a stable disease stare.

The score taking into account the level of CYFRA 21-1 and CA 125 in the sample may be determined by weighted calculation using the determined level(s) of CYFRA 21-1 and/or CA 125.

In embodiments, the score taking into account the level of CYFRA 21-1 and CA 125 may be a binary score and the cut-off score may also be binary. “Binary” means that the score contains two values, e.g. a first value (also referred to as CYFRA 21-1 value) being the level of CYFRA 21-1 or a value derived therefrom and a second value (also referred to as CA125 value) being the level of CA125 or a value derived therefrom. A “value derived therefrom” can, for example, be a value obtained by a mathematical operation. The “value derived therefrom” is preferably direct proportional to the respective level. The values of the binary cut-off score may be obtained by univariate analysis with the respective biomarker, i.e, similar to the procedure of defining a cut-off for a single biomarker as described herein.

Comparing a binary score to a binary cut-off score contains comparing the first value of the determined binary score to the first value of the cut-off binary score and the second value of the determined binary score to the second of the cut-off binary score. In embodiments, if both the first and the second value of the determined binary score are above the first and second value of the binary reference score, respectively, this indicates that the subject does not responds or will not respond to the ongoing NSCLC treatment regime. In embodiments, if one or both of the first and second value of the determined binary score are below the first and second value of the binary reference score, respectively, this indicates that the subject responds or will respond to the ongoing NSCLC treatment regime.

In embodiments, a binary score in which the CYFRA 21-1 value of the determined binary score is above the CYFRA 21-1 cut-off value of the cut-off binary score and the CA 125 value of the determined binary score is above the CA 125 cut-off value of the cut-off binary score indicates that the subject does not responds or will not respond to the ongoing NSCLC treatment regime.

In embodiments, a binary score the CYFRA 21-1 value and/or the CA 125 value of the determined binary score is/are below the CYFRA 21-1 cut-off value and/or the CA 125 cut-off value of the cut-off binary score indicates that the subject responds or will respond to the ongoing NSCLC treatment regime.

Accordingly, the method according to the second aspect may be a method for assessing whether a subject suffering from non-small cell lung carcinoma (NSCLC) responds or will respond to an ongoing NSCLC treatment regime:
a) determining the level of CYFRA 21-1 and the level of CA 125 in a sample obtained from the subject;
aa) determining a binary score comprising a first and a second value and taking into account the level of CYFRA 21-1 determined in a) in the first value and the level of CA 125 determined in a) in the second value;
b) comparing the binary score taking into account the determined level of CYFRA 21-1 and the determined level of CA 125 to a binary cut-off score, said binary cutoff score comprising a CYFRA 21-1 cut-off value and a CA 125 cut-off value, wherein the first value of the determined binary score is compared to the CYFRA 21-1 cut-off value and the second value of the determined binary score is compared to the CA 125 cut-off value;
c) assessing whether the subject based on the comparison in b) responds or will response to the ongoing NSCLC treatment regime,
   wherein the subject from which the sample is obtained previously classified (or diagnosed) to have a stable disease stage.

The method according to the third aspect may further comprise determining the level of CEA in the sample. In these embodiments, the comparing step comprises comparing a score taking into account the determined level of CYFRA 21-1 and/or the determined level of CA 125 and the determined level of CEA to a cut-off score.

Accordingly, in one embodiment of the third aspect of the invention provided is an in vitro method for assessing whether a subject suffering from non-small cell lung carcinoma (NSCLC) responds to an ongoing NSCLC treatment regime, said method comprising:
a) determining the level of CYFRA 21-1, the level of CA 125 and the level of CEA in a sample obtained from the subject;
aa) determining a score taking into account the level of CYFRA 21-1, the level of CA 125 and the level of CEA in the sample; and
b) comparing the score determined in aa) the to a cut-off score,
   wherein the subject is previously classified to have a stable disease (e.g. by imaging such as CT).

In embodiments, the method according to the third aspect may further comprise obtaining the information whether the subject suffers from non-small cell lung carcinoma of subtype squamous cell carcinoma (SCC-NSCLC) or of subtype adenocarcinoma (ADC-NSCLC). Preferably, the obtained information is based on histological data.

As demonstrated in the appended examples, using scores taking into account CYFRA 21-1 and CA 125 levels (and optionally CEA), the further inclusion of information regarding the histological subtype of the NSCLC (adenocarcinoma or SCC) into the score, e.g. by including interaction terms between the determined biomarkers and the histology type in the mathematical formula for calculating the score, the performance in assessing disease progression risk of SD patients was further increased. Consequently, also the performance in assessing treatment response is increased.

In embodiments, the method of the third aspect comprises obtaining information about the NSCLC histological subtype (adenocarcinoma or SCC) and the comparing step comprises comparing a score taking into account the level of the determined biomarker (i.e. CYFRA 21-1 and/or CA 125 and optionally CEA) and the histological NSCLC subtype. In embodiments, the score takes the histological subtype into account by one or more interaction terms between the histological NSCLC subtype and one or more of the determined biomarkers.

Accordingly, in one embodiment of the third aspect, provided is an in vitro method for assessing whether a subject suffering from non-small cell hung carcinoma (NSCLC) responds to an ongoing NSCLC treatment regime, said method comprising:
a) determining the level of CYFRA 21-1 and the level of CA 125 in a sample obtained from the subject;
aa) obtaining information regarding the histological NSCLC subtype from which the subject suffers;
aaa) determining a score taking into account: i) the level of CYFRA 21-1 and the level of CA 125 in the sample; and ii) the histological NSCLC subtype from which the subject suffers (e.g. in form of interaction terms between the biomarker levels and the histological subtype); and
b) comparing the score determined in aaa) the to a cut-off score,
   wherein the subject is previously classified to have a stable disease (e.g. by imaging such as CT).

In another embodiment of the third aspect, provided is an in vitro method for assessing whether a subject suffering from non-small cell lung carcinoma (NSCLC) responds to an ongoing NSCLC treatment regime, said method comprising:
a) determining the level of CYFRA 21-1, the level of CA 125 and the level of CEA in a sample obtained from the subject;
aa) obtaining information regarding the histological NSCLC subtype from which the subject suffers;
aaa) determining a score taking into account: i) the level of CYFRA 21-1, the level of CA 125 and the level of CEA in the sample; and ii) the histological NSCLC subtype from which the subject suffers (e.g. in form of interaction terms between the biomarker levels and the histological subtype); and
b) comparing the score determined in aaa) the to a cut-off score,
   wherein the subject is previously classified to have a stable disease (e.g. by imaging such as CT).

In embodiments of the method according to the third aspect, the method may further comprise providing the information obtained by the method, i.e. the information whether the subject responds to the ongoing treatment regime to the subject and/or a physician. This information may be used to support further treatment decisions, e.g. to maintain or modify the ongoing treatment regime.

In a fourth aspect, the present invention provides a method of treatment of a subject suffering from NSCLC. The method of treatment preferably comprises the steps of one of the methods according to the first, second or third aspect of the invention. All embodiments disclosed herein elsewhere for the methods of the first, second and third aspect apply to the method of the fourth aspect mutatis mutandis. The method of treatment according to the fourth aspect of the invention additionally comprises continuing to treat the patient with the ongoing treatment regime or modifying the treatment regime based on the outcome of the assessment with the methods of the first, second or third aspect.

Accordingly, the method according to the fourth aspect of the invention may comprise (i) assessing whether a subject suffering from non-small cell lung carcinoma (NSCLC) responds to an ongoing treatment regime by a method as defined in any one of the embodiments of the third aspect of the invention; or (i)′ assessing whether an ongoing treatment regime of said subject should be maintained or modified as defined in any one of the embodiments of the second aspect of the invention; and (ii) continuing to treat the patient with the ongoing treatment regime or modifying the treatment regime based on the results of the assessment in (i) or (i)′.

In embodiments of the method according to the fourth aspect. “continuing the treatment regime” means that the ongoing treatment regime is maintained, i.e. that further treatment cycles take place as planned or recommended by treatment guidelines.

The ongoing treatment regime may in principle be any treatment regime for NSCLC. Guidelines for NSCLC are known in the art (S3-Leitlinie Prävention, Diagnostik, Therapie und Nachsorge des Lungenkarzinoms Langversio; and NCCN Clinical Practice Guidelines in Oncology (NCCN Guidelines®) for NSCLC in their most recent versions at the date of filing). In embodiments, the “ongoing treatment regime” may be selected from the group consisting of: chemotherapy, target therapy, immunotherapy, radiotherapy and any combinations thereof.

The appended Examples have demonstrated that the response to various different treatment regimes can be successfully assessed with the methods and uses as described herein.

In specific embodiments, the “ongoing treatment regime” may be a chemotherapy treatment regime.

Specific but non-limiting but exemplary ongoing treatment regimes are disclosed in the appended Examples, specifically in Tables 1a, 1b and 1c.

In embodiments, chemotherapy may be selected from the non-limiting group of: platinum-based chemotherapy and platinum-free chemotherapy.

Platinum-based chemotherapies are therapies comprising administration of a pharmaceutical composition comprising a platinum-based chemotherapeutical. The platinum-based chemotherapeutical may be selected from cisplatin, carboplatin, oxalplatin, nedaplatin, triplatin nitrate, phenathriplatin, picoplatin, satraplatin or any combinations thereof. In a particular embodiment, the platinum-based chemotherapeutical may be selected from carboplatin and cisplatin.

Platinum-free chemotherapy are therapies in which only chemotherapeutic agents other than platinum-based chemotherapeutic drugs are to be administered. Non-limiting examples for chemotherapeutic agents other than platinum-based chemotherapeutic drugs are Etoposid, Docetaxel, Gemcitabin, Paclitaxel, Permetrexed, Vinorelbin and Vincristin.

A targeted therapy is a type of cancer treatment that specifically targets proteins that control how cancer cells grow, divide and/or spread. These therapies thus typically preferentially target cancer cells.

Exemplary but non-limiting targeted therapies are therapies with a tyrosine kinase inhibitor (also referred to as TKI herein). Exemplary but non-limiting examples for tyrosine kinase inhibitors are Afatinib, Alectinib, Crizotinib, Erlotinib and Osimertinib.

Immunotherapies are treatments with an antibody (monospecific or multispecific), antibody-fragment or an antibody-like molecule.

Exemplary but non-limiting immunotherapy agents are: Atezolizumab (Tecentriq®), Durvalumab (Imfinzfi®), Nivolumab (Opdivo®) and Pembrolizumab (Keytruda®). In embodiments, a treatment with an immunotherapy agent may be with a immunotherapy agent selected from Atezolizumab (Tecentriq®), Nivolumab (Opdivo®) and Pembrolizumab (Keytruda®).

In embodiments, immunotherapies may be checkpoint inhibitors such as PD-1 or PD-L1 blockers.

Radiation therapy or radiotherapy, often abbreviated RT, RTx, or XRT, is a therapy using ionizing radiation. Radiotherapy may be given together with chemotherapy, and may be used with curative intent in people with NSCLC who are not eligible for surgery. This form of high-intensity radiotherapy is called radical radiotherapy. A refinement of this technique is continuous hyperfractionated accelerated radiotherapy (CHART), in which a high dose of radiotherapy is given in a short time period. Postoperative thoracic radiotherapy generally should not be used after curative intent surgery for NSCLC. If cancer growth blocks a short section of bronchus, brachytherapy (localized radiotherapy) may be given directly inside the airway to open the passage. Compared to external beam radiotherapy, brachytherapy allows a reduction in treatment time and reduced radiation exposure to healthcare staff. Evidence for brachytherapy, however, is less than that for external beam radiotherapy.

In embodiments of the method according to the fourth aspect, “modifying the treatment regime” includes but is not limited to: (i) adjusting the dose of the treatment (e.g. increasing the dose of the drug), (ii) changing the type of treatment to another type of treatment (e.g. if the ongoing treatment is a chemotherapy, modifying the treatment may be changing the treatment to an immunotherapy). In particular, modifying the ongoing treatment regime may be changing from the specific ongoing treatment regime to any one of the above-mentioned examples of“ongoing treatment regimes”. Expressed in other words, the term “modifying the ongoing treatment regime” may be “adjusting the ongoing treatment regime” or “changing the ongoing treatment regime”.

In a fifth aspect, the present invention relates to the use of CYFRA 21-1 and/or CA 125 as biomarkers) for assessing:
(i) the risk of NSCLC disease progression under an ongoing NSCLC treatment regime for a subject diagnosed for NSCLC;
(ii) whether a subject diagnosed for NSCLC responds to an ongoing NSCLC treatment regime; and/or
(iii) whether for a subject diagnosed for NSCLC an ongoing NSCLC treatment regime is to be maintained or modified.

The subject for which the assessment is made is a subject classified or diagnosed to have a stable disease NSCLC under the ongoing treatment regime.

The use according to the fifth aspect may comprise any of the embodiments as described for the methods according to the first aspect, second aspect, third aspect of the present invention mutatis mutandis.

For example, the use according to the fifth aspect may further comprise using CEA as biomarker for assessing:
(i) the risk of NSCLC disease progression under an ongoing NSCLC treatment regime for a subject diagnosed for NSCLC;
(ii) whether a subject diagnosed for NSCLC responds to an ongoing NSCLC treatment regime; and/or
(iii) whether tor a subject diagnosed for NSCLC an ongoing NSCLC treatment regime is to be maintained or modified.

In embodiments, the use may further comprise taking into account information whether the subject suffers from non-small cell lung carcinoma subtype squamous cell carcinoma (SCC-NSCLC) or adenocarcinoma (ADC-NSCLC), preferably based on histological data.

In a sixth aspect, the present invention relates to a computer-implemented method for assessing
(i) the risk of NSCLC disease progression under an ongoing NSCLC treatment regime for a subject diagnosed for NSCLC;
(ii) whether a subject diagnosed for NSCLC responds to an ongoing NSCLC treatment regime; and/or
(iii) whether for a subject diagnosed for NSCLC an ongoing NSCLC treatment regime is to be maintained or modified, wherein the subject of (i), (ii) and (iii) is classified to have a stable disease.

In a nutshell, the computer-implemented method may be a computer-implemented variant of the methods and uses according to any of the preceding aspects of the invention with the exception that the computer-implemented method does not necessarily involve determining the levels of CYFRA 21-1, CA 125 and/or CEA as well as any other information or data that may be taken into account for calculation of a score.

The computer implemented method may comprise:
a) receiving data comprising the level of CYFRA 21-1 and/or the level of CA 125 and optionally the level of CEA in a sample obtained from a subject and optionally information on the NSCLC subtype of said subject; and
b) processing said data to execute any of the comparing steps as defined in the embodiments of the preceding aspects described herein above.

All embodiments described for the previous aspects and as described herein below apply mutatis mutandis.

A skilled person knows who to establish and configure such a computer-implemented method with routine methods described in the art.

In embodiments the computer-implemented method according to the sixth aspect may further comprise outputting the result of the assessment, i.e. outputting whether:
(i) the subject is at high or low risk of NSCLC disease progression under the ongoing NSCLC treatment regime;
(ii) the subject responds to the ongoing NSCLC treatment regime; and/or
(iii) the subject regime is to be maintained or modified.

The outputting may be achieved with a display.

In a seventh aspect, the present invention relates to a computer program product comprising instructions which, when the program is executed by a computer, cause the computer to carry out any one of the methods and uses as defined in the embodiments according to the first, second, third and fifth aspect of the invention.

All embodiments described for the previous aspects and as described herein below apply mutatis mutandis.

In an eighth aspect, the present invention provides a computer-readable medium comprising instructions which, when executed by a computer, cause the computer to carry out any one of the methods and uses as defined in the embodiments according to the first, second, third and fifth aspect of the invention.

All embodiments described for the previous aspects and as described herein below apply mutatis mutandis.

In a ninth aspect, the present invention provides a data processing system comprising.
(i) a receiving unit configured to receiving data comprising the level of CYFRA 21-1 and/or the level of CA 125 and optionally the level of CEA in a sample obtained from a subject and optionally information on the NSCLC subtype of said subject;
(ii) a processing unit configured to execute any of the comparing steps as defined in the embodiments of the preceding aspects described herein above; and
(iii) optionally an outputting unit configured to output the assessment results.

The assessment results may be the information whether:
(i) the subject is at high or low risk of NSCLC disease progression under the ongoing NSCLC treatment regime;
(ii) the subject responds to the ongoing NSCLC treatment regime; and/or
(iii) the subject regime is to be maintained or modified.

All embodiments described for the preceding aspects and as described herein below apply to the data processing system according to the ninth aspect of the invention mutatis mutandis.

In a tenth aspect, the present invention relates to a kit comprising:
a reagent or a set of reagents for detecting the level of CYFRA 21-1 and/or a reagent or a set of reagents for detecting CA 125 in a sample obtained from a subject.

All embodiments described for the preceding aspects and as described herein below apply to the kit according to the ninth aspect of the invention mutatis mutandis.

The kit is preferably a kit for assessing:
(i) the risk of NSCLC disease progression under an ongoing NSCLC treatment regime for a subject diagnosed for NSCLC;
(ii) whether a subject diagnosed for NSCLC responds to an ongoing NSCLC treatment regime; and/or
(iii) whether for a subject diagnosed for NSCLC an ongoing NSCLC treatment regime is to be maintained or modified, wherein the subject of (i), (ii) and (iii) is a subject diagnosed to have a stable disease (e.g. by CT-based imaging).

In a specific embodiment, the kit comprises a reagent or a set of reagents for detecting the level of CYFRA 21-1 and a reagent or a set of reagents for detecting CA 125 in a sample obtained from a subject.

In embodiments, the kit further comprises a reagent or a set of reagents for detecting the level of CEA in a sample obtained from a subject.

Reagents or set of reagents for detecting CYFRA 21-1 are well known in the art. Exemplary reagents are antibodies or antibody pairs specifically directed to the respective biomarkers. Example for such reagents are the reagents used in the Elecsys® assays used in the appended examples.

In embodiments, the kit may comprise instruction how to assess based on the detected levels of CYFRA 21-1, CA 125 and optionally CEA and/or information on the subtype of NSCLC (i.e. adenocarcinoma or SCC) (i) the risk of NSCLC disease progression under an ongoing NSCLC treatment regime for a subject diagnosed for NSCLC; (ii) whether a subject diagnosed for NSCLC responds to an ongoing NSCLC treatment regime; and/or (iii) whether for a subject diagnosed for NSCLC an ongoing NSCLC treatment regime is to be maintained or modified.

These instructions may comprise cut-offs to which the level(s) need to be compared. In embodiments, the instructions may comprise information on calculation or building for a score (in particular as described elsewhere herein) based on the biomarkers for which detection agents or sets of detection agents are comprised in the kit (i.e. CYFRA 21-1, CA 125 and/or CEA).

In embodiments, the description may comprise a reference to a computer-implemented method (e.g. via a web link), a computer program product, a computer-readable medium, or a data processing system as described herein above.

In an embodiment, the kit may comprise a package insert which specifies that the kit is a kit for assessing:
(i) the risk of NSCLC disease progression under an ongoing NSCLC treatment regime for a subject diagnosed for NSCLC;
(ii) whether a subject diagnosed for NSCLC responds to an ongoing NSCLC treatment regime; and/or
(iii) whether for a subject diagnosed for NSCLC an ongoing NSCLC treatment regime is to be maintained or modified;
wherein the subject of (i), (ii) and (iii) is a subject diagnosed to have a stable disease (e.g. by CT-based imaging).

The following definitions and embodiments apply to all aspects of the invention described herein.

Non-small cell lung carcinoma (NSCLC) is a histological type of lung cancer. Lung cancer is also known as carcinoma of the lung or pulmonary carcinoma, and is a malignant lung tumor characterized by uncontrolled cell growth in tissues of the lung. If left untreated, this growth can spread beyond the lung by process of metastasis into nearby tissue or other parts of the body. Most cancers that start in the lung, known as primary lung cancers, are carcinomas that derive from epithelial cells. The main four histological types of lung cancer are squamous cell carcinoma, adenocarcinoma, large cell carcinoma and small cell carcinoma (SCLC). The first three subtypes are generally referred to as non-small-cell carcinoma (NSCLC) and account for approximately 80% of lung cancer. Diagnosis of the lung tumor is in general based on imaging methods and analysis of biopsy samples. The 2004 World Health Organization (WHO) schema of lung tumors has been the foundation tor lung cancer classification. This incorporated a number of developments, including recognition of lung carcinoma heterogeneity, the introduction of diagnostic immunohistochemical staining (IHC) techniques for the routine diagnosis of some neuroendocrine tumors, and the recognition of newly described entities such as fetal adenocarcinoma, cystic mucinous tumors, and large cell neuroendocrine carcinoma. In 2011, a multidisciplinary expert panel representing the international Association for the Study of Lung Cancer (TASLC), the American Thoracic Society (AlTS), and the European Respiratory Society (IRS) proposed a major revision of the classification system. These changes primarily affect the classification of adenocarcinoma and its distinction from squamous cell carcinoma. The present international standard for classification of tumors by oncologists and pathologists is provided by the “WHO Classification of Tumours of the Lung, Pleura, Thymus and Heart” (Travis et al, 2015, WHO Classification of Tumours, Volume 7, fourth edition). In case of doubt in the context of the present invention, the above standard is to be applied for defining NSCLC and the histological subtypes thereof.

Squamous cell lung carcinoma (SCC) is a type of non-small cell lung cancer formed from reserve cells, i.e. round cells that replaced injured or damaged cells in the lining of the bronchi, the lung's major airways. Squamous cell tumors usually occur in the lung's central portions or in one of the main airway branches. These tumors can form cavities in the lung if they grow to a large size. Making up between 25 and 30 percent of all lung cancers, squamous cell carcinoma can spread to bones, adrenal glands, the liver, small intestine, or brain. The prognosis for an advanced stage of this type of lung cancer is poor. However, five-year survival rates can be as high as 35 to 40 percent for those who have localized lung cancer that is identified and removed in its early stages. These five-year survival rates approach 85 percent for patients under age 30. This type of cancer is almost always caused by smoking. Secondary risk factors include age, family history, and exposure to secondhand smoke, mineral and metal dust, asbestos, or radon.

Histologic diagnosis of squamous cell carcinoma is predicated upon the presence of keratin production by tumor cells and/or intercellular desmosomes (referred to as “intercellular bridges”). Some tumors that are either predominantly spindled (spindle cell variant of pleomorphic carcinoma) or have a characteristic pattern of peripheral palisading may also be classified as squamous cell carcinoma. Historically, most squamous cell carcinoma (60 to 80 percent) arose in the proximal portions of the tracheobronchial tree, through a squamous metaplasia, dysplasia, carcinoma in situ sequence (squamous carcinoma in situ). A minority of cases occur peripherally and may be associated with bronchiectatic cavities or scars. Central and peripheral squamous cell carcinomas may show extensive central necrosis with resulting cavitation. A small subset of central, well differentiated squamous cell carcinoma occur as exophytic, endobronchial, papillary lesions. Patients with this unusual variant of squamous cell carcinoma typically present with persistent cough, recurrent hemoptysis, or relapsing pulmonary infections due to airway obstruction.

Adenocarcinoma is the most common type of lung cancer in contemporary series, accounting for approximately one-half of lung cancer cases. It is a type of lung cancer that forms in mucus-secreting glands throughout the body. Adenocarcinoma is usually found in outer parts of the lung, tends to grow slower than other types of lung cancer, and is more likely to be found before it has spread outside of the lung. It occurs mainly in current or former smokers, but it is also the most common type of lung cancer seen in non-smokers. It is more common in women than in men, and it is more likely to occur in younger people than other types of lung cancer. The increased incidence of adenocarcinoma is thought to be due to the introduction of low-tar filter cigarettes in the 1960s, although such causality is unproven.

The subject for which the assessment in the context of the present invention is provided preferably has NSCLC of subtype adenocarcinoma and/or squamous cell lung carcinoma.

The ongoing treatment regime may in principle be any treatment regime for NSCLC. Guidelines for NSCLC are known in the art (S3-Leitlinie Prävention, Diagnostik, Therapie und Nachsorge des Lungenkarzinoms Langversion 1.0—February 2018 AWMF-Registernunmmer: 020/007OL; and NCCN Clinical Practice Guidelines in Oncology (NCCN Guidelines®) for NSCLC. Version 7.2015. In embodiments, the “ongoing treatment regime” may be selected from the group consisting of: chemotherapy, target therapy, immunotherapy, radiotherapy and any combinations thereof.

The appended Examples have demonstrated that the response to various different treatment regimes can be successfully assessed with the methods and uses as described herein.

In specific embodiments, the “ongoing treatment regime” may be a chemotherapy treatment regime.

Specific but non-limiting but exemplary ongoing treatment regimes are disclosed in the appended Examples, specifically in Tables 1a, 1b and 1c.

In embodiments, chemotherapy may be selected from the non-limiting group of: platinum-based chemotherapy and platinum-free chemotherapy.

Platinum-based chemotherapies are therapies comprising administration of a pharmaceutical composition comprising a platinum-based chemotherapeutical. The platinum-based chemotherapeutical may be selected from cisplatin, carboplatin, oxalplatin, nedaplatin, triplatin nitrate, phenathriplatin, picoplatin, satraplatin or any combinations thereof. In a particular embodiment, the platinum-based chemotherapeutical may be selected from carboplatin and cisplatin.

Platinum-free chemotherapy are therapies in which only chemotherapeutic agents other than platinum-based chemotherapeutic drugs are to be administered. Non-limiting examples for chemotherapeutic agents other than platinum-based chemotherapeutic drugs are Etoposid, Gemcitabin, Docetaxel, Paclitaxel, Permetrexed, Vinorelbin and Vincristin.

A targeted therapy is a type of cancer treatment that specifically targets proteins that control how cancer cells grow, divide and/or spread. These therapies thus typically preferentially target cancer cells.

Exemplary but non-limiting targeted therapies are therapies with a tyrosine kinase inhibitor (also referred to as TKI herein). Exemplary but non-limiting examples for tyrosine kinase inhibitors are Afatinib, Alectinib, Crizotinib. Erlotinib and Osimertinib.

Immunotherapies are treatments with an antibody (monospecific or multispecific), antibody-fragment or an antibody-like molecule.

Exemplary but non-limiting immunotherapy agents are: Atezolizumab (Tecentriq®), Durvalumab (Imfinzi®), Nivolumab (Opdivo®) and Pembrolizumab (Keytruda®). In embodiments a treatment with immunotherapy agents may be with a immunotherapy agent selected from Atezolizumab (Tecentriq®), Nivolumab (Opdivo®) and Pembrolizunab (Keytruda®).

In embodiments, immunotherapies may be checkpoint inhibitors such as PD-1 or PD-L1 blockers.

Radiation therapy or radiotherapy, often abbreviated RT, RTx, or XRT, is a therapy using ionizing radiation. Radiotherapy may be given together with chemotherapy, and may be used with curative intent in people with NSCLC who are not eligible for surgery. This form of high-intensity radiotherapy is called radical radiotherapy. A refinement of this technique is continuous hyperfractionated accelerated radiotherapy (CHART), in which a high dose of radiotherapy is given in a short time period. Postoperative thoracic radiotherapy generally should not be used after curative intent surgery for NSCLC. If cancer growth blocks a short section of bronchus, brachytherapy (localized radiotherapy) may be given directly inside the airway to open the passage. Compared to external beam radiotherapy, brachytherapy allows a reduction in treatment time and reduced radiation exposure to healthcare staff. Evidence for brachytherapy, however, is less than that for external beam radiotherapy.

In preferred embodiments of the aspects of the invention, the ongoing treatment regime may by a first line treatment (i.e. the first treatment, which the subject is subjected to.

“Modifying the NSCLC treatment regime” as used herein may include but is not limited to: (i) adjusting the dose of the treatment (e.g. increasing the dose of the drug), (ii) changing the type of treatment to another type of treatment (e.g. if the ongoing treatment is a chemotherapy, modifying the treatment may be changing the treatment to an immunotherapy). In particular, modifying the ongoing treatment regime may be changing from the specific ongoing treatment regime to any one of the above-mentioned examples of “ongoing treatment regimes”. Expressed in other words, the term “modifying the ongoing treatment regime” may be “adjusting the ongoing treatment regime” or “changing the ongoing treatment regime”. In a preferred embodiment, modifying the ongoing treatment regime may be changing from a first line therapy to a second line therapy.

A treatment regime herein comprises several treatment cycles. A treatment cycle comprises a period of treatment with a treatment type followed by a period of rest (no treatment) that is repeated on a regular schedule. For example, treatment given for one week followed by three weeks of rest is one treatment cycle. When this cycle is repeated multiple times on a regular schedule, it makes up treatment regime comprising several cycles. Preferably, in the context of the present invention a treatment regime comprises three to six treatment cycles (optionally followed by maintenance therapy). Exemplary, a treatment regime may comprise four treatment cycles. A preferred length of a treatment cycle is from 15 days to 27 days, preferably 18 days to 24 days, most preferably 21 days.

In the context of all aspects of the invention, the expression “ongoing treatment regime” refers to a treatment regime administered to the subject at the time the sample was obtained. The treatment regime itself does explicitly not form part of the methods and uses according to any of the first, second, third, fifth and sixth aspect of the invention. The term “ongoing” only implies that the sample, which is subjected to methods of the invention, was obtained at a time the subject was under a treatment regime. Accordingly the methods and uses of the invention except for the method according to the fourth aspect are not practiced on the human body but are mere “in vitro” methods and uses.

Response to a treatment regime in preferred embodiments of the aspects of the present invention means that the subject benefits from the treatment regime. In the context of the invention this may specifically mean that the risk for disease progression (tumor growth, new lesion, formation of metastasis and/or NSCLC caused death) is delayed or avoided.

NSCLC is typically staged in stages I to IV. In the context of all aspects of the invention, the subject from which the sample is obtained may be of any of these stages. In preferred embodiments of the aspects of the invention, the subject may have NSCLC of stage III or IV.

Presently, tumor staging for NSCLC is based on the “The Revised International System for Staging Lung Cancer”. Lung cancer has been categorized considering the information from a clinical database of more than 5,000 patients, was adopted in 2010 by the American Joint Committee on Cancer (AJCC) and the Union Internationale Contre le Cancer.

The T (primary tumor) classification is as follows:
T1=tumor ≤3 cm in size
T2=tumor >3-7 cm in size
T3=tumor >7 cm in size OR multiple tumor nodules in the same lobe
T4=multiple tumor nodules in the same lung buta different lobe

The N/M (metastasis) classification is as follows-:
N0=no regional lymph node metastasis
N1=metastasis in ipsilateral peribronchial and/or ipsilateral hilar lymph nodes and intrapulmonary nodes including involvement by direct extension
N2=metastasis in ipsilateral mediastinal and/or subcranial lymph node(s)
N3=metastasis in contralateral mediastinal, contralateral hilar, ipsilateral or contralateral scalene or supraclavicular lymph node(s)
M0=no distant metastasis
M1=distant metastasis

Tumor stages I to IV are classified in accordance with the above TNM categories as follows:

| | | |
|---|---|---|
| Stage | T1, N0, M0 | T1 = Tumor ≤3 cm in greatest dimension, |
| I | T2a, N0, | surrounded by lung or visceral pleura, without |
| | M0 | bronchoscopic evidence of invasion more proximal |
| | | than the lobar bronchus (i.e., not in the main |
| | | bronchus) |
| | | T2 = Tumor >3 cm but ≤7 cm (T2a: >3 cm but ≤5 |
| | | cm; T2b: >5 cm but ≤7 cm) in greatest dimension, |
| | | or tumor with any of the following features: |
| | | involves main bronchus, ≥2 cm distal to the carina; |
| | | invades visceral pleura (PL1 or PL2); or is |
| | | associated with atelectasis or obstructive |
| | | pneumonitis that extends to the hilar region but does |
| | | not involve the entire lung. |
| | | N0 = No regional lymph node metastasis |
| | | M0 = No distant metastasis |
| Stage | T1, N1, M0 | T1, T2, N0, M0: as above |
| II | T2b, N0, | T3 = Tumor >7 cm or one that directly invades any |
| | M0 | of the following: parietal pleural (PL3) chest wall |
| | T2, N1, M0 | (including superior sulcus tumors), diaphragm, |
| | T3, N0, M0 | phrenic nerve, mediastinal pleura, or parietal |
| | | pericardium or tumor in the main bronchus (<2 cm |
| | | distal to the carina but without involvement of the |
| | | carina) or associated atelectasis or obstructive |
| | | pneumonitis of the entire lung or separate tumor |
| | | nodule(s) in the same lobe |
| | | N1 = Metastasis in ipsilateral peribronchial and/or |
| | | ipsilateral hilar lymph nodes and intrapulmonary |
| | | nodes, including involvement by direct extension |
| Stage | T1, N2, M0 | T1, T2, T3, N1, M0: as above |
| III | T1, N3, M0 | T4 = Tumor of any size that invades any of the |
| | T2, N2, M0 | following: mediastinum, heart, great vessels, |
| | T2, N3, M0 | trachea, recurrent laryngeal nerve, esophagus, |
| | T3, N1, M0 | vertebral body, carina, or separate tumor nodule(s) |
| | T3, N2, M0 | in a different ipsilateral lobe |
| | T3, N3, M0 | N2 = Metastasis in ipsilateral mediastinal and/or |
| | T4, N0, M0 | subcarinal lymph node(s) |
| | T4, N1, M0 | N3 = Metastasis in contralateral mediastinal, |
| | T4, N2, M0 | contralateral bilar, ipsilateral or contralateral |
| | T4, N3, M0 | scalene, or supraclavicular lymph node(s) |
| Stage | Any T, Any | T, N: as above |
| IV | N, M1 | M1 = Distant metastasis (Separate tumor nodule(s) |
| | | in a contralateral lobe tumor with pleural nodules or |
| | | malignant pleural (or pericardial) effusion and/or |
| | | distant metastasis in extrathoracic organs) |

Still more detailed information on the staging may be obtained from “The IASLC Lung Cancer Staging Project: Proposal for Revision of the TNM Stage Groupings in the Forthcoming eighth edition of the TNM Classification for Lung Cancer” (Goldstraw et al JTO 2016; 11:39-51).

The ECOG Scale of Performance Status is a measurement for the impact of the disease on the subjects daily living abilities (known to physicians and researchers as a patient's performance status). It describes a patient's level of functioning in terms of their ability to care for themself, daily activity, and physical ability (walking, working, etc.).

The scale was developed by the Eastern Cooperative Oncology Group (ECOG), now part of the ECOG-ACRIN Cancer Research Group, and published in 1982 (Oken M, et al. Toxicity and response criteria of the Eastern Cooperative Oncology Group. Am J Clin Oncol. 1982: 5:649-655).

The ECOG scale according to this reference is as follows

| | |
|---|---|
| 0 | Fully active, able to carry on all pre-disease performance without |
| | restriction |
| 1 | Restricted in physically strenuous activity but ambulatory and able to |
| | carry out work of a light or sedentary nature, e.g., light house work, |
| | office work |
| 2 | Ambulatory and capable of all selfcare but unable to carry out any |
| | work activities; up and about more than 50% of waking hours |
| 3 | Capable of only limited selfcare; confined to bed or chair more than |
| | 50% of waking hours |
| 4 | Completely disabled; cannot carry on any selfcare; totally confined to |
| | bed or chair |
| 5 | Dead |

In the context of the present invention there is no specific restriction regarding the ECOG score that the subject being assessed (i.e. from whom the sample is derived) may have. In particular embodiments of the aspects of the invention, the subject may have an ECOG score of 0 to 2.

NSCLC patients being under an ongoing treatment regime are typically monitored by imaging based methods such as computer tomography (CT). Based on this monitoring by imaging the tumor response to the treatment can be assessed. On the basis of the tumor imaging results, the patients are typically categorized in one of the following categories: complete response (CR), partial response (PR), progressive disease (PD) and stable disease (SD). Standard criteria for this categorization, such as RECIST 1.1 are available. In the context of the present invention the definitions of the categories are preferably as follows: Partial Response (PR) is assigned to >=30% decrease in tumor size. Progressive Disease (PD) is assigned to >=20% increase in tumor size. Stable Disease is assigned for such cases in that neither sufficient decrease in size to qualify as PR nor sufficient growth to qualify as a PD was found (i.e. <30% decrease in size to <20% increase in size). CR is defined as disappearance of all target lessons. This categorization is based on the response evaluation criteria in solid tumors (RECIST 1.1; see Eisenhauer et al., Eur J Cancer 2009, 45, 228-247, doi:10.1016/j.ejca.2008.10.026).

In context of the invention, the subject may be classified to have stable disease. The classification is preferably by imaging methods such as CT according to the criteria based on RECIST 1.1 as defined above or as defined in RECIST 1.1 (see Eisenhauer et al., Eur J Cancer 2009, 45, 228-247, doi:10.1016/j.ejca.2008.10.026, which is incorporated in its entirety). In the case of a conflict, the RECIST 1.1 criteria prevail. As used herein, the expressions “classified to have stable disease” or “diagnosed to have stable disease” means that the subject's response to the ongoing treatment regime has been assessed, preferably by imaging based (e.g. CT or PET CT) and has been categorized as stable disease according to the above described criteria (preferably RECIST 1.1). In embodiments of the invention (including all aspects of the invention), the subject may be classified to have a stable disease after the second treatment cycle of the ongoing treatment regime. In particular, the subject may be classified to have a stable disease 100 days or less, preferably 80 days or less, preferably 70 days or less, preferably 60 days or less after the 2^{nd} treatment cycle (e.g. 100 days or less, preferably 80 days or less, preferably 70 days or less, preferably 60 days or less after the drug/treatment administration of the 2^{nd} treatment cycle. In preferred embodiments, the subject is classified to have a stable disease based on data (e.g. tumor imaging data such as CT data) obtained at least 10 days, preferably at least 20 days and even more preferably at least 30 days, and most preferably at least 35 days after the start date of the ongoing treatment regime. In embodiments, the subject is classified to have a stable disease based on data (e.g. tumor imaging data such as CT data) obtained at most 150 days, preferably at most 120 days and most preferably at most 108 days after the start date of the ongoing treatment regime. Accordingly, the subject may be classified to have a stable disease based on data (e.g. tumor imaging data such as CT data) obtained 10 days to 150 days after the start date of the ongoing treatment regime, preferably 20 days to 120 days after the start date of the ongoing treatment regime and most preferably 35 days to 108 days after the start date of the ongoing treatment regime.

The “start” or “start date” of the ongoing treatment regime is the day the first treatment of this treatment regime was administered to the subject. For example, the day the drug or drug composition was administered the first time to the subject.

In the context of the present invention, a sample is a sample obtained from a subject (alternatively also referred to as “individual” herein). The subject according to the present invention may be any human or non-human animal, especially a mammal. Thus, the methods and uses described herein are applicable to both human and veterinary disease. Evidently, non-human mammals of particular interest include domestic animals, pets, and animals of commercial value (e.g. domestic animals such as horses) or personal value (e g. pets such as dogs, cats). The method is especially preferred with human subjects, for which diagnostic methods are commonly employed. In a particularly preferred embodiment, the subject from which the sample was obtained is therefore a human.

The “sample” employed in the context of the present invention may be any sample suitable for measuring the biomarkers according to the present invention (i.e. CYFRA 21-1 and/or CA 125 and optionally CEA) and refers to a biological sample obtained for the purpose of evaluation in vitro. It comprises material which can be specifically related to the individual and from which specific information about the individual can be determined, calculated or inferred. A sample can be composed in whole or in part of biological material from the patient (e.g., a solid tissue sample obtained from a lung biopsy). A sample can also be material that has contacted the patient in a way that allows tests to be conducted on the sample, which provides information about the individual. The sample may preferably comprise any body fluid. Exemplary test samples include blood, serum, plasma, urine, saliva, and fluid from the lungs (such as epithelial lining fluid), e.g. obtained by bronchoscopy or broncholavage. The sample may be taken from the subject and used immediate or processed before the determination/measuring. Processing may include purification (e.g. separation such as centrifugation), concentration, dilution, lysis of cellular components, freezing, acidification, conservation etc. Preferred samples are whole blood, serum, and plasma. In a particularly preferred embodiment, the sample is serum or plasma.

Typically, blood-related samples are preferred test samples for use in the context of the present invention. These samples may have been obtained by drawing blood from a vein, usually from the inside of the elbow or the back of the hand. Particularly, in infants or young children, a sharp tool called a lancet may be used to puncture the skin and make it bleed. Accordingly, the blood may be venous blood. The blood may be collected e.g. into a pipette, or onto a slide or test strip. Accordingly, in a preferred embodiment of the present invention, the sample obtained from the individual is a blood sample, particularly selected from the group consisting of serum, plasma and whole blood. Most preferably, the sample is a blood sample, selected from serum or plasma.

The sample obtained from the subject to be assessed in the context of the present invention, may be a sample obtained at a defined time point within the ongoing NSCLC treatment regime the subject is undergoing. In embodiments, the sample is a sample that was obtained within a specific time frame around the day of the medical examination (e.g. imaging such as CT) eventually leading to diagnosis of stable disease. In embodiments, the specific time frame may be from 30 days before the medical examination to 30 days after the medical examination. In a preferred embodiment, the specific time frame may be from 20 days before the medical examination to 30 days after the medical examination. In an even more preferred embodiment, the specific time frame may be from 10 days before the medical examination to 29 days after the medical examination. In embodiments, the sample is a sample obtained after the treatment administration phase of the second cycle of the treatment regime. In preferred embodiments, the sample is a sample obtained from the subject at least 10 days, preferably at least 20 days and most preferably at least 25 days after the start date of the ongoing treatment regime. In embodiments, the sample is a sample obtained from the subject at most 150 days, preferably at most 120 and most preferably at most 108 days after the start date of the ongoing treatment regime. Accordingly, in embodiments, the sample is a sample obtained from the subject 10 days to 150 days after the start date of the ongoing treatment regime, preferably 20 days to 120 days after the start date of the ongoing treatment regime, and most preferably 25 days to 108 days after the start date of the ongoing treatment regime. The “start” or “start date” of the ongoing treatment regime is the day the first treatment of this treatment regime was administered to the subject. For example, the day the drug or drug composition was administered the first time to the subject.

The present invention uses the biomarkers CYFRA 21-1 and/or CA 125 Optionally also CEA may be used.

CYFRA 21-1 belongs to the cytokeratin family. Cytokeratins are structural proteins forming the subunits of epithelial intermediary filaments, which is a major component of the cell cytoskeleton. Twenty different cytokeratin polypeptides with molecule weights ranging from 40 to 70 Kilodaltons (kD) have so far been identified. The type of cytokeratin synthesized by a cell is also affected by the growth and differentiation rate. Due to their specific distribution patterns they are eminently suitable for use as differentiation markers in tumor pathology. CYFRA 21-1 is a fragment of cytokeratin 19, which is a part of cytoskeleton in epithelial cells, and can be found in an overexpressed way in tumors of epithelial origin. Intact cytokeratin polypeptides are poorly soluble, but soluble fragments can be detected in serum (Bodenmueller et al., 1994, Int. J. Biol. Markers 9: 75-81).

CA 125 (cancer antigen 125, carcinoma antigen 125, or carbohydrate antigen 125) also known as mucin 16 or MUC16 is a protein that in humans is encoded by the MUC16 gene. MUC16 is a member of the mucin family glycoproteins. CA 125 is a membrane-associated mucin that possesses a single transmembrane domain. A unique property of CA 125 is its large size. CA125 is more than twice as long as MUC1 and MUC4 and contains about 22,000 amino acids, making it the largest membrane-associated mucin. CA 125 has been shown to play a role in advancing tumorigenisis and tumor proliferation by several different mechanisms.

CEA is a monomeric glycoprotein (molecular weight approx. 180,000 Dalton) with a variable carbohydrate component of approx. 45-60%. CEA, like AFP, belongs to the group of carcinofetal antigens that are produced during the embryonic and fetal period. The CEA gene family consists of about 17 active genes in two subgroups. The first group contains CEA and the Non-specific Cross-reacting Antigens (NCA), the second group contains the Pregnancy-Specific Glycoproteins (PSG). CEA is mainly found in the fetal gastrointestinal tract and in fetal serum. It also occurs in slight quantities in intestinal, pancreatic, and hepatic tissue of healthy adults.

In the context of the present invention when talking about CYFRA 21-1, CA 125 and CEA reference is made to the respective proteins not to nucleic acids encoding the same.

The methods and uses described in the aspects of the invention may comprise the step of determining the level of certain biomarker proteins. Specifically, the level of CYFRA 21-1 and/or CA 125 and optionally CEA may be determined. Determining a level may alternatively be expressed as “measuring a level of a biomarker”.

A variety of methods for measuring a protein biomarker molecule (particularly CYFRA 21-1, CA 125 or CEA) are known in the art and any of these can be used.

Preferably, the biomarker(s) is/are specifically measured from a liquid sample by use of a specific binding agent.

A specific binding agent is, e.g., a receptor for the biomarker or an antibody to the marker. In the context of the invention, the biomnarker molecule(s) (i.e. CYFRA 21-1, CA 125 and/or CEA) is/are measured at the protein level.

Determination of proteins as binding partners of a marker polypeptide can be performed using any of a number of known methods for identifying and obtaining proteins that specifically interact with proteins or polypeptides, for example, a yeast two-hybrid screening system such as that described in U.S. Pat. Nos. 5,283,173 and 5,468,614, or the equivalent. A specific binding agent has preferably at least an affinity of 108 l/mol or even more preferred of at least 109 l/mol for its target molecule. As the skilled artisan will appreciate the term specific is used to indicate that other biomolecules present in the sample do not significantly bind to the binding agent specific for the marker. Preferably, the level of binding to a biomolecule other than the target molecule results in a binding affinity which is only 10% or less, more preferably only 5% or less of the affinity to the target molecule, respectively. A preferred specific binding agent will fulfill both the above minimum criteria for affinity as well as for specificity.

A specific binding agent preferably is an antibody reactive with a biomarker, particularly CYFRA 21-1, CA 125 or CEA. The term antibody refers to a polyclonal antibody, a monoclonal antibody, antigen binding fragments of such antibodies, single chain antibodies as well as to genetic constructs comprising the binding domain of an antibody.

The term “antibodies” includes polyclonal antibodies, monoclonal antibodies, fragments thereof such as F(ab′)2, and Fab fragments, as well as any naturally occurring or recombinantly produced binding partners, which are molecules that specifically bind a CYFRA 21-1, CA 125 or CEA polypeptide. Any antibody fragment retaining the above criteria of a specific binding agent can be used. Antibodies are generated by state of the art procedures, e.g., as described in Tijssen (Tijssen, P., Practice and theory of enzyme immunoassays. Elsevier Science Publishers B.V., Amsterdam (1990), the whole book, especially pages 43-78). In addition, the skilled artisan is well aware of methods based on immunosorbents that can be used for the specific isolation of antibodies. By these means the quality of polyclonal antibodies and hence their performance in immunoassays can be enhanced (Tijssen, P., supra, pages 108-115).

For the achievements as disclosed in the present invention polyclonal antibodies raised in e.g. goats, rats, rabbits or guinea pigs, as well as monoclonal antibodies can be used. Since monoclonal antibodies can be produced in any amount required with constant properties, they represent ideal tools in development of an assay for clinical routine.

For determining the level of CYFRA 21-1, CA 125 or CEA or any other protein biomarker the sample obtained front an individual may be incubated with the specific binding agent for the marker in question under conditions appropriate for formation of a binding agent marker-complex. Such conditions need not be specified, since the skilled artisan without any inventive effort can easily identify such appropriate incubation conditions. The amount of binding agent marker-complex is measured and used in the methods and uses of the invention. As the skilled artisan will appreciate there are numerous methods to measure the amount of the specific binding agent marker-complex all described in detail in relevant textbooks (cf., e.g., Tijssen P., supra, or Diamandis, E. P. and Christopoulos, T. K. (eds.), Immunoassay, Academic Press. Boston (1996)).

Particularly, monoclonal antibodies to the markers (CYFRA 21-1, CA 125 and CEA) may be used in quantitative (amount or concentration of the markers is determined) immunoassays.

Preferably, the marker in question is detected in a sandwich type assay format. In such assay a first specific binding agent is used to capture the marker in question on the one side and a second specific binding agent (e.g. a second antibody), which is labeled to be directly or indirectly detectable, is used on the other side. The second specific binding agent may contain a detectable reporter moiety or label such as an enzyme, dye, radionuclide, luminescent group, fluorescent group or biotin, an electrochemiluminescence label (e.g. a Ruthenium based electrochemiluminescence label) or the like. Any reporter moiety or label could be used with the methods disclosed herein so long as the signal of such is directly related or proportional to the quantity of binding agent remaining on the support after wash. Preferred is the use of an electrochemiluminescence label (e.g. a Ruthenium based electrochemiluminescence label). The amount of the second binding agent that remains bound to the solid support is then determined using a method appropriate for the specific detectable reporter moiety or label. For radioactive groups, scintillation counting or autoradiographic methods are generally appropriate. Antibody-enzyme conjugates can be prepared using a variety of coupling techniques (for review see, e.g., Scouten, W. H., Methods in Enzymology 135:30-65, 1987). Spectroscopic methods can be used to detect dyes (including, for example, colorimetric products of enzyme reactions), luminescent groups and fluorescent groups. Biotin can be detected using avidin or streptavidin, coupled to a different reporter group (commonly a radioactive or fluorescent group or an enzyme). Enzyme reporter groups can generally be detected by the addition of substrate (generally for a specific period of time), followed by spectroscopic, spectrophotometric or other analysis of the reaction products. Standards and standard additions can be used to determine the level of antigen in a sample, using well known techniques.

As described above, there are a variety of methods for measuring CYFRA 21-1 levels. An assay for “CYFRA 21-1” specifically measures a soluble fragment of cytokeratin 19 as present in the circulation. The measurement of CYFRA 21-1 is typically based upon two monoclonal antibodies (Bodenmueller et al., 1994, Int. J. Biol. Markers 9: 75-81). Commercially available products for measuring CYFRA 21-1 include the Elecsys® CYFRA 21-1 assay from Roche Diagnostics, Cytokeratin 19 fragment (CYFRA 21-1) immunoradiometric assay kit (Cisbo Assays, Codolet, France), ELISA Kit for Cytokeratin Fragment Antigen 21-1 (Wuhan USCN Business Co., Ltd., China) and the ARCHITECT CYFRA 21-1 assay (Abbott. Wiesbaden, Germany). In the CYFRA 21-1 assay from Roche Diagnostics, Germany, the two specific monoclonal antibodies (KS 191 and BM 19.21) are used and a soluble fragment of cytokeratin 19 having a molecular weight of approx. 30,000 Daltons is measured. Preferably, CYFRA 21-1 levels are measured with Elecsys® using Roche product number 11820966122 according to the manufacturer's instructions.

For the measurement of CA 125 levels, there exists a variety of commercially available assays. Commercially available products for measuring CA 125 levels include, for example, the Elecsys® CA 12511 assay from Roche Diagnostics and the CA 12511 Assay (Siemens Healthineers. Erlangen, Germany). Preferably, CA 125 levels are measured using the Elecsys® CA125 II assay (Product Number 11776223322).

Also for the measurement of CEA levels, there exits a variety of assays. Commercially available products for measuring CEA include the ADVIA Centaur® CEA immunoassay (Siemens Healthineers. Erlangen. Germany) and the ARCHITECT CEA assay (Abbott, Wiesbaden. Germany). Preferably. CEA is measured using “Elecsys® CEA” (Material No: 11731629322, Roche Diagnostics, Ltd, Rotkreuz, Switzerland), which is an electro-chemiluminescence immunoassay (ECLIA) for the quantitative determination of CEA.

The step of determining the level of a marker may be carried out as follows: The sample and optionally calibrator and/or control may be contacted with the binding agent (which could be immobilized, e.g. on a solid phase) under conditions allowing the binding of the agent to the marker, Unbound binding agents may be removed by a separation step (e.g. one or more washing steps). A second agent (e.g. a labeled agent) may be added to detect the bound binding agent to allow binding to and quantification of the same. Unbound second agent may be removed. The amount of the second binding agent which is proportional to the amount of the marker may be quantified, e.g. based on the label. Quantification may be done based on e.g. a calibration curve constructed for each assay by plotting measured value versus the concentration for each calibrator. The concentration or amount of marker in the sample may be then read from the calibration curve.

Determining the “level” of a biomarker (e.g. CYFRA 21-1, CA 125 or CEA) in a sample means that the amount or concentration of the respective biomarker molecule in the sample is determined. The amount of a substance may be an absolute amount (e.g. give in mass) or a standards-defined quantity that measures the size of an ensemble of elementary entities, such as atoms, molecules, electrons, and other particles. It is sometimes referred to as chemical amount. The international System of Units (SI) defines the amount of substance to be proportional to the number of elementary entities present. The SI unit for amount of substance is the mole. It has the unit symbol mol. The concentration of a substance is the amount of a constituent divided by the total volume of a mixture. Several types of mathematical description can be distinguished: mass concentration, molar concentration, number concentration, and volume concentration. The term concentration can be applied to any kind of chemical mixture, but most frequently it refers to solutes and solvents in solutions. The molar (amount) concentration has variants such as normal concentration and osmotic concentration.

The “level of a biomarker” as used herein relates to a level of said biomarker based on a measurement of the amount or concentration in a sample of the subject at a defined time point during an ongoing treatment. The “level of a biomarker” as used herein does specifically not relate to a change in a biomarker level over two time points. It is a gist of the present invention that surprisingly single biomarker values give raise to a better disease progression risk assessment (and consequently treatment response assessment/prognosis) than a change in biomarker levels which were previously used.

In the context of the present invention, “a score taking into account . . . ” may be determined. This means that the individual parameters taken into account are mathematically combined, e.g. by weighted calculation. Alternatively, a binary score may be built, as described herein above. The individual parameters taken into account may be the determined CYFRA 21-1 level in the sample and/or the determined CA 125 level in the sample. Optionally, also the CEA level in the sample may be taken into account. The levels may be used as such or may be mathematically transformed (e.g. by log transformation such as log 2 or log 10 transformation). In a preferred embodiment, level(s) of the biomarkers CYFRA 21-1 and/or CA 125 and optionally CEA may be log transformed (i.e. the log 2 or log 10 value(s) is/are calculated) for determining the score. Optionally, also the information whether the subject has NSCLC of histological subtype adenocarcinoma or SCC may be taken into account, preferably by including an interaction term between the biomarkers taken into account and the histological subtype.

In embodiments of the invention, the score may be obtained by weighted calculation of the amount or concentration of the marker molecule(s) in the samples. This means that the markers may be given different weighting than the other. Exemplary, when the score takes into account the level (amount or concentration) of CYFRA 21-1 ([CYFRA 21-1]) and the level (amount or concentration) of CA 125 ([CA 125]) in the sample, the score may be calculated by the following equation:

Score=a*[CYFRA21-1]+b*[CA125],

wherein a and b represent the weighting factors. Preferably, the weighting factors have been obtained by analyzing a reference population (e.g. any reference population as defined in the context of the cut-off value below). A suitable procedure is described in the Examples.

In embodiments of the invention the score may besides biomarkers also take into account the information regarding the histology type (i.e. adenocarcinoma or SCC) of the NSCLC. The information regarding the tumor histology is preferably taken into account by using an interaction term (Vatcheva, K. P., et al. Epidemiology (Sunnyvale, Calif.) 6.1, 2015) between the tumor histology information and each biomarker to account for the association between histology and a respective biomarker.

An example for a model formula including three biomarkers and the interaction term between the biomarker and the histology of the Cox-regression is:

h(t)=h₀(t)*exp(β₁*log 2(CYFRA21-1)+β₂*log 2(CYFRA21-1)×SCC+β₃*log 2(CA125)+β₄*log 2(CA125)*SCC+β₅*log 2(CEA)+β₆*log 2(CEA)*SCC)

With t representing the survival time, h(t) representing the hazard function, the coefficients β₁, . . . , β₆ measuring the impact of the covariates, SCC representing the histology squamous cell carcinoma and the biomarkers CYFR21-1, CA125 and CEA which are in the above formula log 2 transformed (log 2 transformation is preferred but not mandatory).

For patients without SCC the formula reduces as SCC is set to 0:

h(t)=h₀(t)*exp(β₁*log 2(CYFRA21-1)+β₃*log 2(CA125)+β₅*log 2(CEA))

For patients with SCC the whole formula is taken into account as SCC is set to 1. Similar to the model including multiple biomarkers a score can be built based, e.g. on the risk prediction of the Cox regression model.

In embodiments, the score taking into account the level of CYFRA 21-1 and CA 125 may be a binary score and the cut-off score may also be binary. “Binary” means that the score contains two values, e g. a first value (also referred to as CYFRA 21-1 value) being the level of CYFRA 21-1 or a value derived therefrom and a second value (also referred to as CA125 value) being the level of CA 125 or a value derived therefrom, A “value derived therefrom” can, for example, be a value obtained by a mathematical operation. The “value derived therefrom” is preferably direct proportional to the respective level. The values of the binary cut-off score may be obtained by univariate analysis with the respective biomarker, i.e, similar to the procedure of defining a cut-off for a single biomarker as described herein. Comparing a binary score to a binary cut-off score involves comparing the first value of the determined binary score to the first value of the cut-off binary score and the second value of the determined binary score to the second of the cut-off binary score.

A skilled person in the art is aware that the score and a corresponding cut-off score can be optimized based on a reference population (e.g. any as defined in the context of the determination of a cut-off below).

For example, the score can be determined for each sample of the reference population and subsequent the median or a suitable cut-off of the combined values can be chosen. In a suitable cohort one may calculate a Cox's proportional hazards regression model to evaluate the effect of CYFRA 21-1 and/or CA 125 and optionally CEA as predictor variables on the survival.

The regression coefficients delivered by the Cox's proportional hazards regression model for CYFRA 21-1 and/or CA 125 and optionally CEA and optionally the interaction terms between the biomarkers and the histology information can then be used to calculate the score for each patient in the cohort as weighted combination of biomarkers and potentially histology information.

As the Cox's proportional hazards model is a relative risk model, the prediction of each patient is calculated relative to the sample average (e.g. based on a reference population as specified elsewhere herein). This means that the parameter levels of each patient are adjusted by the mean of the population. The score could then be calculated like the following:

Score=β₁*(log 2(CYFRA21-1)−mean(log 2(CYFRA21-1)))+β₂(log 2(CYFRA21-1)−mean(log 2(CYFRA21-1)))*SCC+β₃*(log 2(CA125)−mean(log 2(CA125)))+β₄*(log 2(CA125)−mean(log 2(CA125)))SCC+β₅*(log 2(CEA)−mean(log 2(CEA)))+β₆*(log 2(CEA)−mean(log 2(CEA)))*SCC

For scores the cut-off can be calculated using a reference population, e.g. by determining the median score or an optimized cut-off score (see below).

Risk groups may then be compared by Kaplan-Meier curve for illustration purposes.

Cox's proportional hazards regression (Cox, David R 1972 Journal of the Royal Stalisrical Society, Breslow. N E. 1975 International/Statistical Review) is a typical method in survival analysis to examine the relationship of the survival distribution to covariates. The Cox model is usually written in terms of the hazard model formula shown below. This model gives an expression for the hazard at time t for an individual with a given specification of a set of explanatory variables denoted by the X. X presents a collection of predictor variables that is being modeled to predict an individual's hazard. The Cox model formula says that the hazard at time t is the product of two quantities. h0(t) is called the baseline hazard function. The second quantity is the exponential expression e to the linear sum of βᵢXᵢ where the sum is over the p explanatory X variables (Gail, M. et al 1996 Statistics for Biology and Health).

h(t,X)=h0(t)e^{Σ}^{ᵢ₌₁}^{^{p}}βiXi

Patient categorization may involve Kaplan-Meier (KM) survival curves. A KM curve is a non-parametric statistic used to estimate the survival function from lifetime data (Kaplan. E. L; Meier, P 1958 J. Amer. Statist. Assn), KM curves are not smooth functions, but rather step-wise estimates. The X-axis shows the serial time of the survival duration. The cumulative probability that a member from a given population will have a lifetime exceeding time, t is seen on the Y-axis. Cumulative probabilities for an interval are calculated by multiplying the interval survival rates up to that interval. (Rich J T et al 2010 Otolaryngol Head Neck Surg). KM curves are typically applied for grouping patients into categories.

The Kaplan-Meier estimator is a statistic, and several estimators are used to approximate its variance. One of the most common such estimators is Greenwood's formula:

$$( \hat{S} ( t ) ) = {\hat{S} ( t )}^{2} {\sum }_{{t}_{i} \leq t} \frac{{d}_{i}}{{n}_{i} ( {α}_{i} - {d}_{i} )}$$

$$( \hat{S} ( t ) ) = {\hat{S} ( t )}^{2} {\sum }_{{t}_{i} \leq t} \frac{{d}_{i}}{{n}_{i} ( {n}_{i} - {d}_{i} )}$$

In some cases, one may wish to compare different Kaplan-Meier curves. This may be done by several methods including the log rank test or the Cox proportional hazards test.

The expressions “comparing the determined level of CYFRA 21-1 to a CYFRA 21-1 cut-off value”. “comparing the determined level of CA 125 to a CA 125 cut-off value”, “comparing the score taking into account . . . to a cut-off score” are merely used to further illustrate what is obvious to the skilled artisan anyway.

In accordance with the present invention, the respective cut-offs may in particular be defined by using a reference population. Preferably, the reference population may be composed of NSCLC patients under an ongoing treatment regime (as defined herein elsewhere) and being classified to have stable disease (as defined herein elsewhere). The reference population shall preferably comprise SD patients being at high risk for disease progression and SD patients being at low risk for disease progression. In embodiments, the patients of the reference population may have NSCLC of subtype adenocarcinoma or SCC. In embodiments, the patients of the reference population may have NSCLC of stage III or IV. In embodiments, the ECOG of the patients of the reference population may be from 0 to 2. In an embodiment the reference population may be a patient group as defined in the section “Study population” in the appended Examples. Disease progression (i.e. survival time and time at which disease progression was detected) must be known for the patients of the reference population. Similarly the relevant parameter for which a cut-off is to be defined, i.e. the CYFRA 21-level in the sample, the CA 125 level and/or the score must be known for the patients of the reference population. The respective cut-off may then be defined as that level of CY FRA 21-1. CA 125 and/or that score dividing the reference population of SD patients best into two groups, a first group with increased disease progression risk and a further group with decreased progression risk. The respective cut-offs may be selected such that the desired separation between the two groups is best. The quality of the separation can, for example, be measured by the hazard ratio. For example, a marker level (amount or concentration) in a patient sample can be compared to a level known to be associated with a high risk or with a low risk patient. It is within the skills of the practitioner to choose an appropriate cut-off or cut-off value for the markers or scores herein. As also clear to the skilled artisan, the levels of the respective biomarkers established in a control will be dependent on the assay used. Preferably, samples from 100 or more well-characterized individuals from the appropriate reference population are used to establish a cut-off. Also preferred the reference population may be chosen to consist of at least 20, 30, 50, 100, 200, 500 or 1000 individuals. Preferably, the biomarker levels are obtained at the time point the sample of the subject analyzed by the present invention are obtained (as defined herein elsewhere).

In embodiments of the invention, the level of CYFRA 21-1, CA 125, or the score according to the invention corresponding to the median level of the respective measure in the reference population may be selected as cut-off.

In a preferred embodiment of the invention, respective cut-offs may be optimized cut-offs. Such optimized cut offs may be determined in that all quantiles from 0.2 to 0.8 by steps of 0.05 of the biomarker or score value were tested for their performance to split the patients into two groups and the hazard ratio and log-rank p-value for each split was calculated. The quantile with the lowest log-rank p-value (highest hazard ratio) may then be chosen as the optimized cut-off. Optimized cutoff values may be also chosen by alternative measures of risk discrimination between high and low risk patients group whereby the suited measure will be chosen by the skilled practitioner.

In general, the disclosure is not limited to the particular methodology, protocols, and reagents described herein because they may vary. Further, the terminology used herein is for the purpose of describing particular embodiments only and is not intended to limit the scope of the present disclosure. As used herein, the singular forms “a”. “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the disclosure.

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include the respective terms also in plural, unless the content clearly dictates otherwise.

The following figures and examples are provided to aid the understanding of the present invention, the true scope of which is set forth in the appended claims. It is understood that modifications can be made in the procedures set forth without departing from the spirit of the invention.

### DESCRIPTION OF THE FIGURES

The following Figures are provided to aid the understanding of the present invention, the true scope of which is set forth in the appended claims. It is understood that modifications can be made in the procedures set forth without departing from the spirit of the invention.

FIG. 1: Progression-free survival (A) and overall survival (B) in patients with partial response or stable disease at the first CT scan after the second cycle stratified by CT scan results.

FIG. 2: Progression-free survival in patients with stable disease at the first CT scan after the second cycle in those with adenocarcinoma stratified by CYFRA 21-1 (A), squamous cell carcinoma stratified by CA 125 (B), adenocarcinoma or squamous cell carcinoma stratified by CYFRA 21-1 and CA 125 (C), and adenocarcinoma or squamous cell carcinoma stratified by CYFRA 21-1, CA 125 and CEA (D), above or below median. Biomarker combinations by score (referred to as Pred in the Figures) in (C) and (D) take into account interaction terms between each biomarker and the histology. No baseline correction for biomarker levels was used.

FIG. 3: Progression-free survival (A) and overall survival (B) in patients with stable disease at the first CT scan after the second cycle in those with adenocarcinoma or squamous cell carcinoma stratified into high- and low-risk groups by CYFRA 21-1, CA 125 and CEA, above or below median. The model for the biomarker combinations takes into account interaction terms between each biomarker and the histology. No baseline correction for biomarker levels was used.

FIG. 4: Progression-free survival (A) and overall survival (B) in patients with stable disease at the first CT scan after the second cycle in those with adenocarcinoma or squamous cell carcinoma stratified into high- and low-risk groups by CYFRA 21-1, CA 125 and CEA, above or below optimized cut-off. The model for the biomarker combinations takes into account interaction terms between each biomarker and the histology. No baseline correction for biomarker levels was used.

### EXAMPLES

The following examples are provided to aid the understanding of the present invention, the true scope of which is set forth in the appended claims. IT is understood that modifications can be made in the procedures set forth without departing from the spirit of the invention.

### Example 1: Assessment of the Prognostic Value of a Serum Biased Biomarkers in Patients with NSCLC

To study whether blood based tumor biomarkers have predictive value in monitoring treatment success and disease progression prognosis as such or in addition to imaging based analyses, a clinical study was conducted involving parallel state-of-the art imaging based tumor staging/treatment monitoring and parallel measurement of seven pre-selected biomarker candidates (CEA, ProGRP, NSE, CYFRA 21-1, SCC, CA 15-3, and CA 125).

The cohort of this clinical study includes patients ≤18 years of age with previously untreated Stage III or IV NSCLC (adenocarcinoma or squamous cell carcinoma (SCC) histology) and Eastern Cooperative Oncology Group (ECOG) performance score 0-2.

Of the 387 NSCLC patients, 265 patients received first-line treatment, did not have progressive disease before treatment Cycle 2 (Cycle 2) and had computed tomography (CT) data available 25 days to 108 days (median 44 days) after treatment start.

At the first CT after Cycle 2, 230 patients did not have progressive disease (100 patients had stable disease [SD] and 130 patients had a partial response [PR]). For 228 of these patients (100 patients with SD and 128 patients with PR) complete biomarker results, i.e. levels of CEA, ProGRP, NSE, CYFRA 21-1, SCC, CA 15-3, and CA 125 were available.

Exclusion criteria were: inability to obtain blood samples, history of secondary malignancy, severe co-morbidities, any type of tumor-directed pre-treatment (some patients received radiotherapy defined first-line treatment), pregnancy or breastfeeding.

Patients received as first line therapy chemotherapy (75.1% of all patients), tyrosine kinase inhibitors and/or immune checkpoint inhibitors at the discretion of the treating physician.

Patients with NSCLC (adenocarcinoma or SCC) and available CT scan data after the second treatment cycle of the first line therapy were used for the biomarker analysis (see below) Patients who had documented disease progression before the first CT scan were excluded from the analysis with the biomarker data.

Patient demographics and treatment schemes of the analyses population are summarized in Tables 1a, 1b and 1c, below.

TABLE 1a
Patient demographics and treatment schemes (analyses population);
All values n (%), unless otherwise stated. Adeno, adenocarcinoma;
CT, computed tomography; ECOG, Eastern Cooperative Oncology
Group, ICI, immune checkpoint inhibitor; PR, partial response;
SCC, squamous cell carcinoma; SD, stable disease; TKI,
tyrosine kinase inhibitor; UICC, Union for International
Cancer Control.

| | Patients with SD at first CT after Cycle 2 | | | | | |
|---|---|---|---|---|---|---|
| | Adeno | | SCC | | All | |
| | (n = 76) | | (n = 24) | | (N = 100) | |
| Mean age (standard | 67.3 | (8.9) | 67.6 | (8.2) | 67.3 | (8.7) |
| deviation) | | | | | | |
| Male sex | 47 | (61.8) | 22 | (91.7) | 69 | (69.0) |
| Smoking status | | | | | | |
| Smoker | 26 | (34.2) | 8 | (33.3) | 34 | (34.0) |
| Ex-smoker | 42 | (55.3) | 16 | (66.7) | 58 | (58.0) |
| Never smoked | 8 | (10.5) | — | | 8 | (8.0) |
| UICC stage | | | | | | |
| III | 16 | (21.1) | 11 | (45.8) | 27 | (27.0) |
| IV | 60 | (79.0) | 13 | (54.2) | 73 | (73.0) |
| ECOG performance | | | | | | |
| status | | | | | | |
| 0 | 37 | (48.7) | 13 | (54.2) | 50 | (50.0) |
| 1 | 38 | (50.0) | 11 | (45.8) | 49 | (49.0) |
| 2 | 1 | (1.3) | — | | 1 | (1.0) |
| Treatment until first | | | | | | |
| CT after Cycle 2 | | | | | | |
| Platinum-based | 48 | (63.2) | 23 | (95.8) | 71 | (71.0) |
| combination | | | | | | |
| Platinum-free | 6 | (7.9) | — | | 6 | (6.0) |
| TKI ± radiotherapy | 8 | (10.5) | — | | 8 | (8.0) |
| ICI alone or in | 11 | (14.5) | 1 | (4.2) | 12 | (12.0) |
| combination | | | | | | |

| | | | | | |
|---|---|---|---|---|---|
| No treatment | 3 | (4.0) | — | 3 | (3.0) |

TABLE 1b
Patient demographics and treatment schemes (analyses population);
All values n (%), unless otherwise stated. Adeno, adenocarcinoma;
CT, computed tomography; ECOG, Eastern Cooperative Oncology
Group, ICI, immune checkpoint inhibitor; PR, partial
response; SCC, squamous cell carcinoma; SD, stable disease;
TKI tyrosine kinase inhibitor; UICC, Union for International
Cancer Control.

| | Parents with PD at first CT after Cycle 2 | | | | | |
|---|---|---|---|---|---|---|
| | Adeno | | SCC | | All | |
| | (n = 27) | | (n = 8) | | (N = 35) | |
| Mean age (standard | 62.3 | (9.0) | 66.0 | (5.3) | 63.1 | (8.4) |
| deviation) | | | | | | |
| Male sex | 18 | (66.7) | 4 | (50.0) | 22 | (62.9) |
| Smoking states | | | | | | |
| Smoker | 15 | (55.6) | 4 | (50.0) | 19 | (54.3) |
| Ex-smoker | 10 | (37.0) | 3 | (37.5) | 13 | (37.1) |
| Never smoked | 2 | (7.4) | 1 | (12.5) | 3 | (8.6) |
| UICC stage | | | | | | |
| III | 1 | (3.7) | 2 | (25.0) | 3 | (8.6) |
| IV | 26 | (96.3) | 6 | (75.0) | 32 | (91.4) |
| ECOG performance | | | | | | |
| status | | | | | | |
| 0 | 14 | (51.9) | 3 | (37.5) | 17 | (48.6) |
| 1 | 13 | (48.2) | 5 | (62.5) | 18 | (51.4) |
| 2 | — | | — | | — | |
| Treatment until first | | | | | | |
| CT after Cycle 2 | | | | | | |
| Platinum-based | 20 | (74.1) | 8 | (100) | 28 | (80.0) |
| combination | | | | | | |

| Platinum-free | — | | — | — | |
|---|---|---|---|---|---|
| TKI ± radiotherapy | 1 | (3.7) | — | 1 | (2.9) |
| ICI alone or in | 5 | (18.5) | — | 5 | (14.3) |
| combination | | | | | |
| No treatment | 1 | (3.7) | — | 1 | (2.9) |

TABLE 1c
Patient demographics and treatment schemes (analyses population);
All values n (%), unless otherwise stated. Adeno, adenocarcinoma;
CT, computed tomography; ECOG, Eastern Cooperative Oncology
Group; ICI, immune checkpoint inhibitor; PR, partial response:
SCC, squamous cell carcinoma; SD, stable disease; TKI,
tyrosine kinase inhibitor, UICC, Union for International
Cancer Control.

| | Patients with PR at first CT after Cycle 2 | | | | | |
|---|---|---|---|---|---|---|
| | Adeno | | SCC | | All | |
| | (n = 90) | | (n = 38) | | (N = 128) | |
| Mean age (standard | 65.3 | (10.5) | 67.7 | (7.8) | 66.0 | (9.8) |
| deviation) | | | | | | |
| Male sex | 43 | (47.8) | 31 | (81.6) | 74 | (57.8) |
| Smoking status | | | | | | |
| Smoker | 28 | (31.1) | 16 | (42.1) | 44 | (34.4) |
| Ex-smoker | 45 | (50.0) | 21 | (55.3) | 66 | (51.6) |
| Never smoked | 17 | (18.9) | 1 | (2.6) | 18 | (14.1) |
| UICC stage | | | | | | |
| III | 18 | (20.0) | 23 | (60.5) | 41 | (32.0) |
| IV | 72 | (80.0) | 15 | (39.5) | 87 | (68.0) |
| ECOG performance | | | | | | |
| status | | | | | | |
| 0 | 43 | (47.8) | 21 | (55.3) | 64 | (50.0) |
| 1 | 47 | (52.2) | 17 | (44.7) | 64 | (50.0) |
| 2 | — | | — | | — | |
| Treatment until first | | | | | | |
| CT after Cycle 2 | | | | | | |
| Platinum-based | 53 | (58.9) | 36 | (94.7) | 89 | (69.5) |
| combination | | | | | | |
| Platinum-free | 3 | (3.3) | 1 | (2.6) | 4 | (3.1) |
| TKI ± radiotherapy | 20 | (22.2) | 1 | (2.6) | 21 | (16.4) |

| | | | | | |
|---|---|---|---|---|---|
| ICI alone or in | 9 | (10.0) | — | 9 | (7.0) |
| combination | | | | | |
| No treatment | 5 | (5.6) | — | 5 | (3.9) |

### Study Objectives

The main objective was to assess the predictive value of serum biomarkers in monitoring treatment success and the prognosis for disease progression. Specifically, it was assessed whether patients with stable disease (SD) based on CT can be further differentiated into risk groups (e.g. responders with good prognosis) based on serum biomarker levels. This analysis was conducted after the first two cycles of treatment. The measures used for the assessment were overall survival (OS) and progression free survival (PFS), each of them individually analyzed.

### Treatment Efficacy Assessment by State of the Art CT Imaging

The first CT scan after Cycle 2 of first-line treatment was set as the analysis time point for all patients (maximum time between the second cycle treatment and the first CT after Cycle 2 was 60 days). The tumor response on CT was defined by the response evaluation criteria in solid tumors (RECIST 1.1: see Eisenhauer et al., Eur J Cancer 2009, 45, 228-247. doi:10.1016/j.ejca.2008.10.026). Partial Response (PR) was assigned to >=30% decrease in size. Progressive Disease (PD) was assigned to >=20% increase in size. Stable Disease was assigned for such cases were neither sufficient decrease in size to qualify as PR nor sufficient growth to qualify as a PD was found (i.e. <30% decrease in size to <20% increase in size).

Progression-free survival (PFS) time and overall survival (OS) time were calculated from treatment start date. PFS was defined as follows:
Progression=Patient has at least one progression (i.e. PD according to RECIST) observed or died;
No progression=Patient has no progression and was alive at study completion;
PFS time for Patients with Progression=Date of progression (detected)−Therapy start date+1 (in days);
PFS time for patients that died during study=Death date−Therapy start date+1 (in days);
PFS time for patients without progression event=Last contact date−Therapy start date+1 (in days).

OS time was defined as follows:
OS time for patients that died during the study=Death date−Therapy start date+1 (in days);
OS time for patients alive after last contact=Last contact date−Therapy start date+1 (in days).

### Sample Collection and Biomarker Evaluation

Venous blood samples were collected from each patient at baseline (i.e. before treatment start) and each routine visit (usually at each treatment cycle (approx. 21 days each)), if possible. The samples for the analysis at the first CT after the 2^{nd} treatment cycle were taken in a range of 10 days before to the CT to 29 days after the CT. Calculated from the day of the treatment start the samples were taken 25 to 108 days after that date. Serum samples were stored as 500 μl aliquots below −70° C.

In these serum samples biomarker levels were measured at a later timepoint. Specifically, electrochemiluminescence immunoassays (ECLIA) for in vitro diagnostics protein biomarkers CEA, ProGRP, NSE, CYFRA 21-1, SCC, CA 15-3, and CA 125 were conducted on Cobas® systems from Roche Diagnostics Centralized and Point of Care Solutions (CPS) according to the manufacturer's guidelines.

### Statistical Analysis

Patient demographics and disease characteristics were summarized using descriptive statistics. Risk prediction for progression (PFS or OS) was compared between CT response and biomarker values using Cox regression models and Kaplan-Meier curves.

Prognostic models were based on response at first CT after Cycle 2 (PR versus SD), biomarker values at first CT after Cycle 2 and biomarker change between baseline and first CT (baseline correction) after Cycle 2, respectively. Biomarker values were log 2 transformed. For univariate models (including only one biomarker) a cutoff based on the value of the biomarker is used to split the patients in a low and a high risk group, e.g. the median of the biomarker is used. For illustration the cut-offs of the univariate models were backtransformed to the original biomarker scale for the Kaplan-Meier curves. For models including more than one biomarker a score was built by weighted linear combination based on the linear predictor of the Cox regression model. To split the patients in a low and a high risk group e.g. the median of this score was used equally to the univariate biomarker.

Further, in alternative, for models including more than one biomarker a binary-score was built based on the combination of the risk groups of the univariate models of each included biomarker. In brief, similar to the univariate models (including one biomarker) a cut-off based on the on the value of each individual biomarker was defined (e.g. the median of the biomarker levels). If both biomarker levels were above the respective biomarker cut-offs forming pan of the binary score cut-off, the patients were categorized to the high risk group. If a single biomarker was below the respective cut-off; i.e. one of the levels of the binary score was below the corresponding cut-off value of the binary score cut-off, the patients were categorized to the low risk group.

Prognostic models were evaluated using hazard ratios (HRs) and C-Indexes. The C-Index is a non-parametric estimator of the proportion of all patient pairs for which model prediction and observed outcome are concordant and is therefore a global evaluation criteria for the Cox regression model. A C-Index of 1 corresponds to the best model prediction and a C-Index of 0.5 represents a random prediction. A hazard ratio describes the risk between patient groups, e.g. a hazard ratio of 2 means that the risk of a patient in the high risk group is two times higher compared to a patients in the low risk group.

In biomarker-based risk prediction models, patients of the SD group were separated into two risk groups based using either the median biomarker/score value as cut-off or an optimized cut-off. The optimized cut-off was determined in that all quantiles from 0.2 to 0.8 by steps of 0.05 of the biomarker/score value were tested for the performance to split the patients into two groups and the hazard ratio and log-rank p-value for each split was calculated. The quantile with the lowest log-rank p-value was chosen as the optimized cut-off.

Cox proportional hazard models were based on a single biomarker or combinations of two or three biomarkers. For combined population of adenocarcinoma and SCC (histology information), the models also include an interaction term (Vatcheva, K. P., et al. Epidemiology (Sunnyvale, Calif.) 6.1, 2015) between said histology information and each biomarker to account for the association between histology and a respective biomarker. An example for a model formula including three biomarkers and the interaction term between the biomarker and the histology of the Cox-regression is:

h(t)=h₀(t)*exp(β₁*log 2(BM₁)+β₂*log 2(BM₁)×SCC+β₃*log 2(BM₂)+β₄*log 2(BM₂)*SCC+β₅*log 2(BM₃)+β₆*log 2(BM₃)*SCC)

With representing the survival time, h(t) representing the hazard function, the coefficients β₁, . . . , β₆ measuring the impact of the covariates, BM₁, . . . , BM₃ representing the biomarkers and SCC representing the histology squamous cell carcinoma. For patients without SCC the formula reduces as SCC is set to 0:

h(t)=h₀(t)*exp(β₁*log 2(BM₁)+β₃*log 2(BM₂)+β₅*log 2(BM₃))

For patients with SCC the whole formula is taken into account as SCC is set to 1.

Similar to the model including multiple biomarkers a score was built based on the risk prediction of the Cox regression model.

### Results of Study

a) Prognostic Value of State-of-the Art First CT Scan after Second Treatment Cycle

First, the prognostic value of the first CT scan after the second treatment cycle in predicting progression-free survival (PFS) or overall survival (OS) was assessed. Monitoring by CT is the current state of the art in assessing NSCLC progression and treatment response.

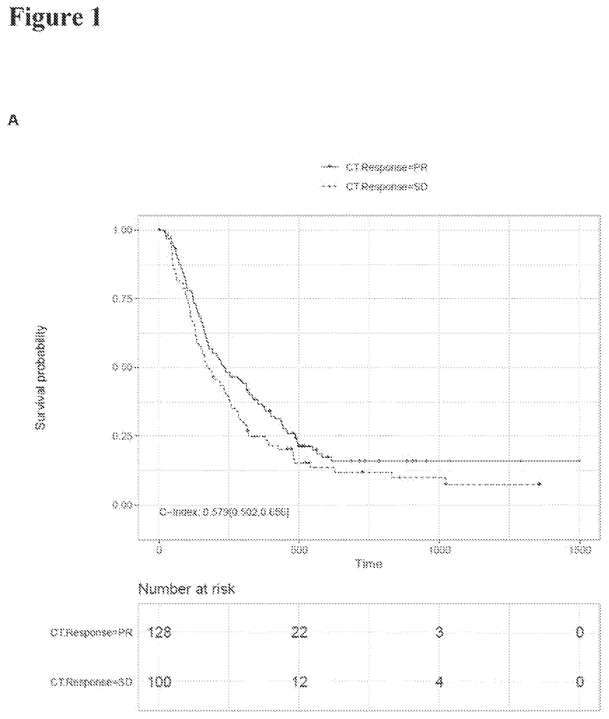

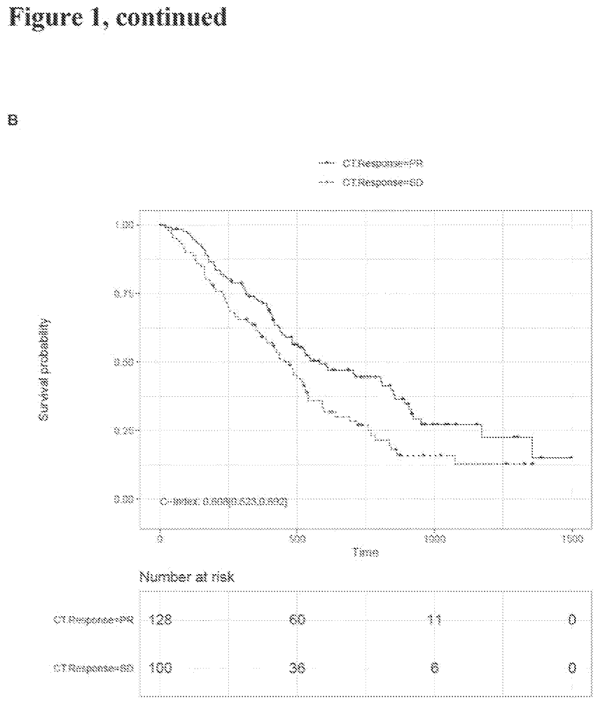

Patients identified as having PR or SD at the first CT scan had similar risk of progression (see FIG. 1A), suggesting that the first CT scan had poor prognostic performance for PFS in patients deriving clinical benefit from the ongoing treatment regime, i.e. patients of the PR and SD groups.

The specific results regarding PFS are as follows:
Adenocarcinoma or SCC: Hazard ratio (HR)=1.326 (p-value=0.055), C-index=0.579.
Adenocarcinoma: HR=1.482 (p-value=0.025), C-Index: 0.616.
SCC: HR=0.992 (p-value=978), C-Index: 0.507.

The first CT scan also showed poor prognostic performance for OS, with rates of OS largely similar in patients with PR and SD (FIG. 1B).

The specific results regarding OS are:
Adenocarcinoma or SCC: HR=1.517 (p-value=0.012), C-index=0.608.
Adenocarcinoma: HR=1.784 (p-value=0.005), C-Index 0.656 SCC: 0.924 (p-value=0.786), C-Index 0.527

### b) Prognostic Value of Cancer Biomarkers Using Non-Baseline Corrected Single Measurement Values

The prognostic value of individual cancer biomarkers (CA 125, CA 15-3, CEA, CYFRA 21-1, NSE. SCC and proGRP), or combinations of cancer biomarkers, in predicting PFS or OS were assessed in patients with SD using single protein biomarker levels in serum without baseline correction (i.e. absolute biomarker level without taking the biomarker concentration before treatment into account).

### Analyses Based on Median Biomarker Cut-Off

In a first set of analyses the biomarker data (non-baseline corrected) were analyzed by splitting SD patients into two groups at the median value for each biomarker; i.e. using the median of the absolute biomarker value or the median of the combined biomarker value (score) as cut-off. The performance of single biomarkers (univariate analyses) and of several biomarkers combined to a score (multivariate analyses) was assessed.

The results for the read-out progression-free survival are summarized in Table 2, below:

TABLE 2
Results of univariate and combination analyses of the prognostic values
of CYFRA 21-1, CA 125, CEA, CA15-3, NSE, SCC and ProGRP for progression-
free survival in patients with stable disease at the first CT scan after
the second cycle. Higher C-index indicates better performance of the
Cox regression model, higher HR indicates better performance in separating
the SD population in high and low risk populations.

| | | | | | | | Adeno + SCC | |
|---|---|---|---|---|---|---|---|---|
| | | | | | | | with | |
| | | | | | Adeno + SCC | | interaction term | |
| | Adeno (n = 76) | | SCC (n = 24) | | (n = 100) | | (n = 100) | |
| | C- | HR | C- | HR | C- | HR | C- | HR |
| | index | median^{a} | index | median^{a} | Index | median^{a} | index | median^{a} |
| Univariate | | | | | | | | |
| analysis | | | | | | | | |
| CYFRA 21-1 | 0.689 | 2.197 | 0.606 | 2.878 | 0.667 | 2.293 | 0.671 | 2.405 |
| CA 125 | 0.626 | 1.897 | 0.680 | 4.087 | 0.619 | 1.707 | 0.632 | 2.492 |
| CEA | 0.597 | 1.547 | 0.509 | 1.213 | 0.561 | 1.270 | 0.585 | 1.503 |
| CA15-3 | 0.557 | 1.141 | 0.563 | 1.087 | 0.554 | 1.162 | 0.559 | 1.189 |
| NSE | 0.535 | 1.147 | 0.506 | 1.043 | 0.528 | 1.019 | 0.537 | 1.141 |
| SCC | 0.520 | 1.219 | 0.610 | 1.306 | 0.538 | 1.229 | 0.545 | 1.103 |
| proGRP | 0.507 | 0.834 | 0.498 | 0.821 | 0.507 | 0.826 | 0.527 | 1.498 |
| Combination | | | | | | | | |
| analysis | | | | | | | | |
| CYFRA 21-1 + | 0.689 | 2.633 | 0.688 | 5.354 | 0.666 | 2.758 | 0.678 | 2.910 |
| CA 125 | | | | | | | | |
| CYFRA 21-1 + | 0.708 | 2.790 | 0.695 | 5.354 | 0.674 | 2.412 | 0.702 | 2.372 |
| CA 125 + CEA | | | | | | | | |

^{a}Patients stratified according to biomarker value above or below median.
Adeno, adenocarcinoma; HR, hazard ratio; SCC, squamous cell carcinoma.

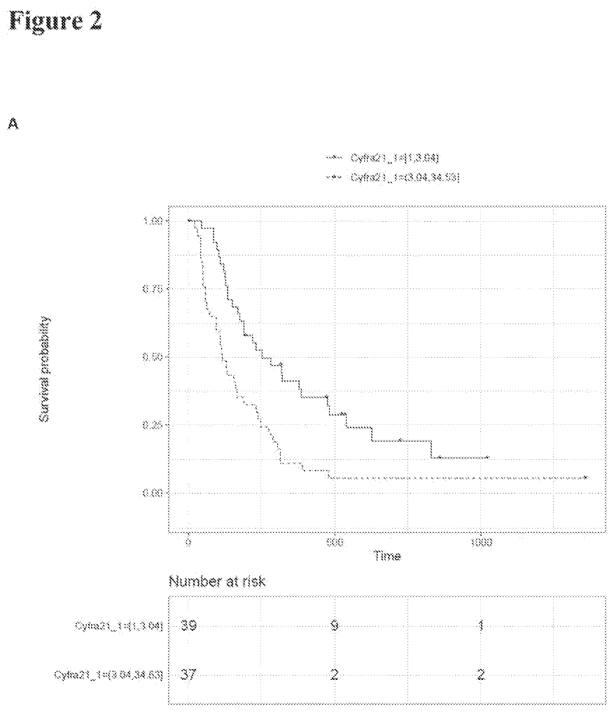

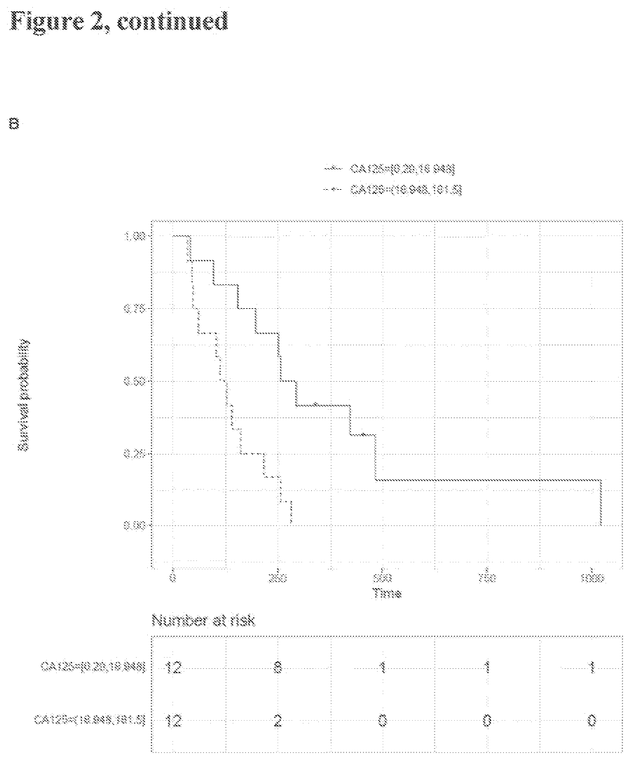

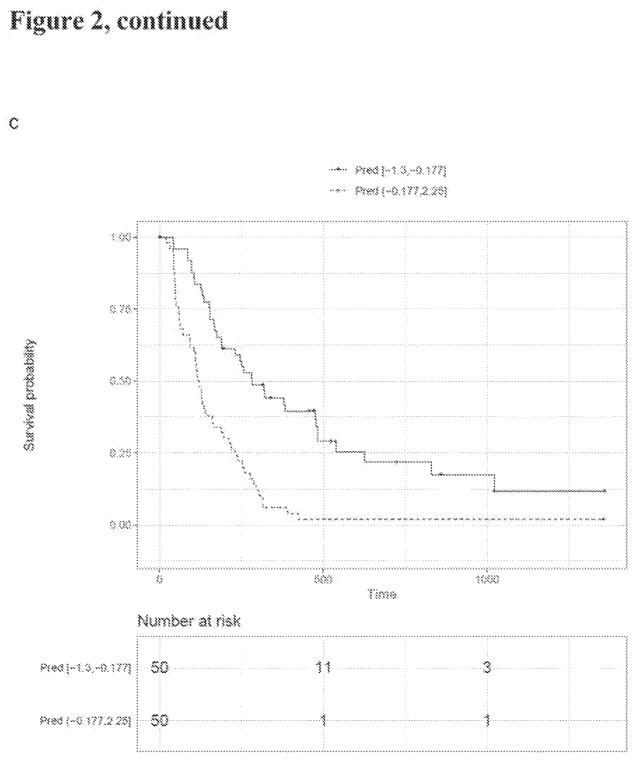

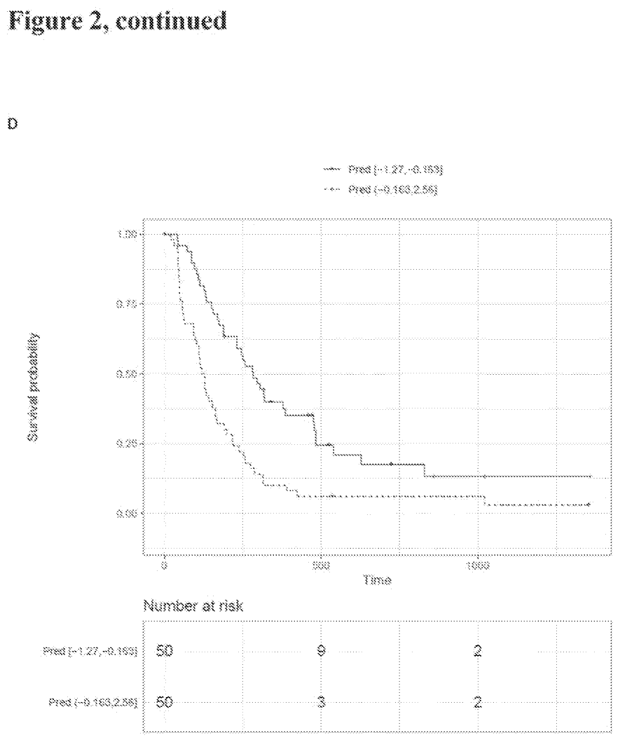

Of the single biomarkers tested, CYFRA 21-1 had the highest prognostic value for adenocarcinoma, in patients with SD (see Table 2 and FIG. 2A) as indicated by C-index and HR. A combination of CYFRA 21-1 and CA 125 showed an improved performance in separating the groups of high and low risk for disease progression, as indicated by a higher HR. The best performance was achieved by a combination analysis (score) involving CYFRA 21-1, CA 1-25 and CEA.

For SCC patients, CA 125 had the highest prognostic value in patients with SD as single biomarker (see Table 2 and FIG. 2B) as indicated by C-index and HR. A combination of CYFRA 21-1 and CA 125 showed an improved performance in C-index and HR. The best performance was achieved by a combination analysis (score) involving CYFRA 21-1, CA 125 and CFA.

For the combined population of adenocarcinoma and SCC, a combination of CYFRA 21-1 and CA 125 (using an interaction term) had a greater prognostic value than either biomarker alone in patients with SD according to RECIST (see Table 2 and FIG. 2C). Building a score taking into account CYFRA 21-1. CA 125, CEA and in addition an interaction term between the biomarkers and the histology (SCC or adenocarcinoma),) the prognostic performance of the combination for adenocarcinoma and SCC in the patient cohort with SD NSCLC could be further increased (see Table 2):

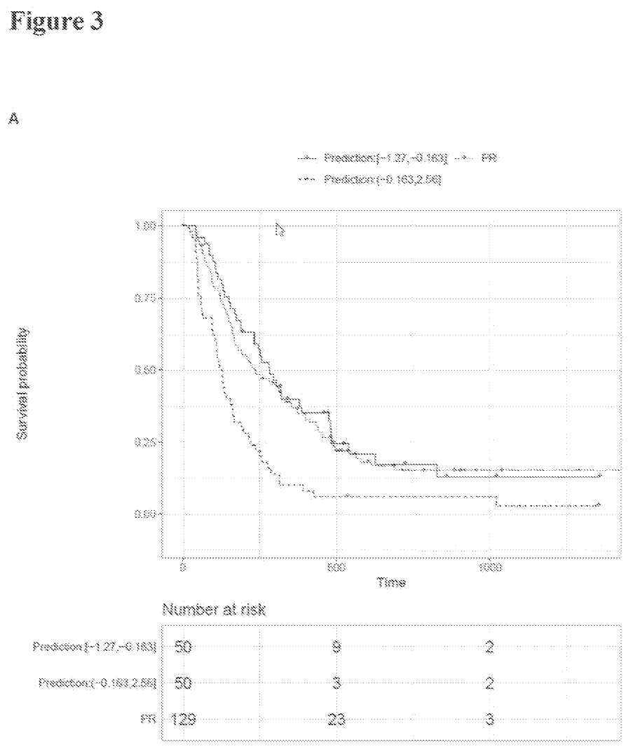

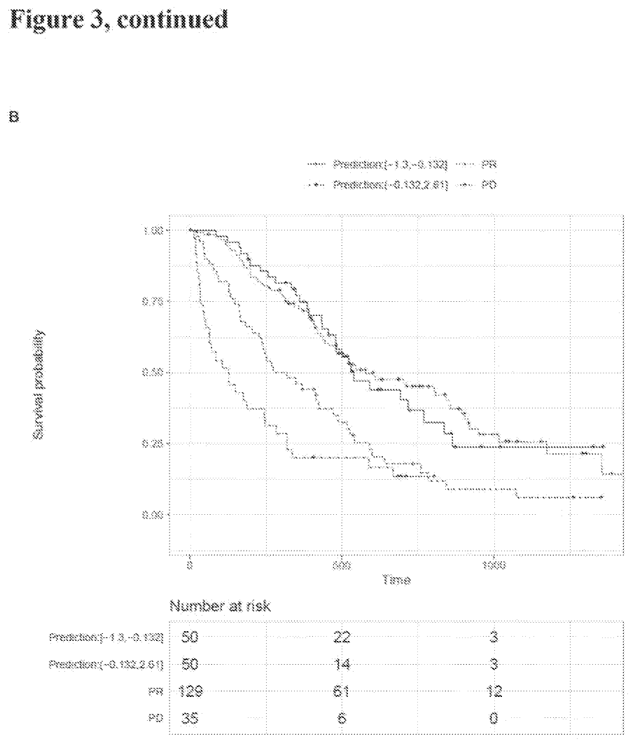

Notably, Patients with SD, and a lower risk of progression or death based on the individual biomarkers CYFRA 21-1 or CA 125 or the combination model including CYFRA 21-1, CA 125 and CEA (and optionally an interaction term), had a similar probability for progression compared with patients with PR as indicated by hazard ratios (HR) (FIG. 3, Table 3). To calculate those hazard ratios, a variable was created which is “PR” for patients with PR, “low risk” for SD patients in the low risk group and “high risk” for SD patients in the high risk group. A further Cox model including this covariate was fitted, this results in the H R between each combination of the three groups (“low risk” vs. “high risk”, “low risk” vs. PR and “high risk” vs. PR).

TABLE 3
Results of univariate and combination analyses of the prognostic
values of CYFRA 21-1, CA 125 and CEA for progression-free
survival in patients with stable disease at the first
CT scan after the second cycle. A HR near to 1 indicates
a similar risk for progression between a patient with
SD in the low risk group and a patient with PR.

| | | | | Adeno + |
|---|---|---|---|---|
| | | | | SCC with |
| | | | Adeno + | interaction |
| | Adeno | SCC | SCC | term |
| | (n = 76 | (n = 24) | (n = 100) | (n = 100) |
| | HR low risk | HR low risk | HR low risk | HR low risk |
| | vs. PR | vs. PR | vs. PR | vs. PR |
| Univariate | | | | |
| analysis | | | | |
| CYFRA 21-1 | 0.972 | 1.482 | 1.096 | 1.130 |
| CA 125 | 0.895 | 1.621 | 0.953 | 1.081 |
| CEA | 0.848 | 1.138 | 0.852 | 0.919 |
| CA15-3 | 0.727 | 1.027 | 0.809 | 0.828 |
| NSE | 0.725 | 1.045 | 0.761 | 0.807 |
| SCC | 0.740 | 1.106 | 0.827 | 0.783 |
| proGRP | 0.609 | 0.895 | 0.681 | 0.920 |
| Combination | | | | |
| analysis | | | | |
| CYFRA 21-1 + | 1.059 | 1.642 | 1.155 | 1.189 |
| CA 125 | | | | |
| CYFRA 21-1 + | 1.056 | 1.642 | 1.102 | 1.107 |
| CA 125 + CEA | | | | |

^{a}Patients stratified according to biomarker value above or below median.
Adeno, adenocarcinoma; HR, hazard ratio; SCC, squamous cell carcinoma.

For PFS, the HR for high versus low risk patients (based on the score taking into account CYFRA 21-1 CA 125, CEA and the histology interaction term) amounts to 2,372 (p-value<0.001), indicating that the risk for disease progression is significantly higher for high risk patients according to the biomarker model (see Table 2). The HR for PR versus SD in the low risk based on this score amounts to 1.107 (p-value 0.594), indicating that the SD patients categorized as low risk for disease progression by the biomarker model have a comparable risk for disease progression than PR patients according to imaging (FIG. 3A and Table 3). Similar analyses made for the first read out PFS have also been made for the second read-out OS.

The data on OS (see Table 4) confirmed the findings based on the PFS readout.

Again, a robust prognostic separation of patients at high risk for disease progression/death and low risk for disease progression/death could be achieved based on the same biomarkers and scores.

For OS, the HR for high versus low risk patients (based on the score taking into account CYFRA 21-1 CA 125, CEA and the histology interaction term) amounts to 2.091 (p-value 0.002), again confirming that also for this read out the separation between high and low risk works well using the biomarker model (see Table 4). The HR for PR versus low risk based on this score amounts to 0.960 (p-value 0.853), confirming that the low risk group identified by the biomarker model has a comparable risk to the PR patient group (see Table 5). The HR for PD versus high risk amounts to 1.760 (FIG. 3B.

TABLE 4
Results of univariate and combination analyses of the prognostic values of
CYFRA 21-1, CA 125 and CEA for overall survival in patients with stable disease
at the first CT scan after the second cycle. Higher C-index indicates better
performance of the Cox regression model, higher HR indicates better performance
in separating the SD population in high and low risk populations.

| | | | | | | | Adeno + SCC | |
|---|---|---|---|---|---|---|---|---|
| | | | | | | | with | |
| | | | | | Adeno + SCC | | interaction term | |
| | Adeno (n = 76) | | SCC (n = 24) | | (n = 100) | | (n = 100) | |
| | C- | HR | C- | HR | C- | HR | C- | HR |
| | index | median^{a} | index | median^{a} | Index | median^{a} | index | median^{a} |
| Univariate | | | | | | | | |
| analysis | | | | | | | | |
| CYFRA 21-1 | 0.713 | 2.771 | 0.609 | 1.305 | 0.694 | 2.363 | 0.692 | 2.357 |
| CA 125 | 0.659 | 2.561 | 0.617 | 3.827 | 0.640 | 2.040 | 0.649 | 2.573 |
| CEA | 0.559 | 1.119 | 0.512 | 0.877 | 0.545 | 1.117 | 0.545 | 1.394 |
| CA15-3 | 0.583 | 1.424 | 0.672 | 1.400 | 0.599 | 1.575 | 0.599 | 1.549 |
| NSE | 0.544 | 1.373 | 0.528 | 1.206 | 0.542 | 1.130 | 0.525 | 1.246 |
| SCC | 0.528 | 1.248 | 0.540 | 1.123 | 0.532 | 1.366 | 0.541 | 1.303 |
| proGRP | 0.517 | 1.175 | 0.533 | 0.878 | 0.522 | 1.123 | 0.516 | 1.081 |
| Combination | | | | | | | | |
| analysis | | | | | | | | |
| CYFRA 21-1 + | 0.729 | 3.110 | 0.617 | 1.673 | 0.705 | 2.541 | 0.703 | 2.540 |
| CA 125 | | | | | | | | |
| CYFRA 21-1 + | 0.715 | 2.740 | 0.665 | 1.527 | 0.703 | 2.354 | 0.698 | 2.091 |
| CA 125 + CEA | | | | | | | | |

^{a}Patients stratified according to biomarker value above or below median.
Adeno, adenocarcinoma; HR, hazard ratio; SCC, squamous cell carcinoma.

TABLE 5
Results of univariate and combination analyses of the prognostic
values of CYFRA 21-1, CA 125 and CEA for overall survival in patients
with stable disease at the first CT scan after the second cycle.
A HR near to 1 indicates a similar risk for progression between
a patient with SD in the low risk group and a patient with PR.

| | | | | Adeno + |
|---|---|---|---|---|
| | | | | SCC with |
| | | | Adeno + | Interaction |
| | Adeno | SCC | SCC | term |
| | (n = 76) | (n = 24) | (n = 100) | (n = 100) |
| | HR low risk | HR low risk | HR low risk | HR low risk |
| | vs. PR | vs. PR | vs. PR | vs. PR |
| Univariate | | | | |
| analysis | | | | |
| CYFRA 21-1 | 0.906 | 1.211 | 0.989 | 1.015 |
| CA 125 | 0.900 | 1.842 | 0.932 | 1.031 |
| CEA | 0.610 | 1.014 | 0.706 | 0.787 |
| CA15-3 | 0.675 | 1.218 | 0.811 | 0.815 |
| NSE | 0.677 | 1.263 | 0.715 | 0.747 |
| SCC | 0.639 | 1.199 | 0.785 | 0.765 |
| proGRP | 0.623 | 1.038 | 0.706 | 0.701 |
| Combination | | | | |
| analysis | | | | |
| CYFRA 21-1 + | 0.978 | 1.272 | 1.032 | 1.045 |
| CA 125 | | | | |
| CYFRA 21-1 + | 0.916 | 1.259 | 0.994 | 0.960 |
| CA 125 + CEA | | | | |

^{a}Patients stratified according to biomarker value above or below median.
Adeno, adenocarcinoma; HR, hazard ratio; SCC, squamous cell carcinoma.

In sum, the above results demonstrate that CYFRA 21-1 and CA 125 absolute levels as such, a score combining the absolute levels of the two biomarkers and in particular a score taking into account CYFRA 21-1, CA 125 and CEA absolute levels can provide additional guidance to the first CT scan after the second treatment cycle in patients with indeterminate CT response, by differentiating those with SD into high and low risk groups. This was validated by using PFS and OS. Further, we have demonstrated that the biomarker score can be further improved by taking into account an interaction term based on tumor histology (SCC or adenocarcinoma) and optimizing the cut-off.

### Analyses Based Optimized Biomarker Cut-Off

The analysis using the model/score based on CYFRA 21-1, CA 125 and CEA and using the interaction term was repeated with an optimized cut-off Specifically, the cut-off was optimized to obtain the maximum difference in risk between the biomarker groups by keeping 20% of the patients in each of the two risk groups (high and low risk group).

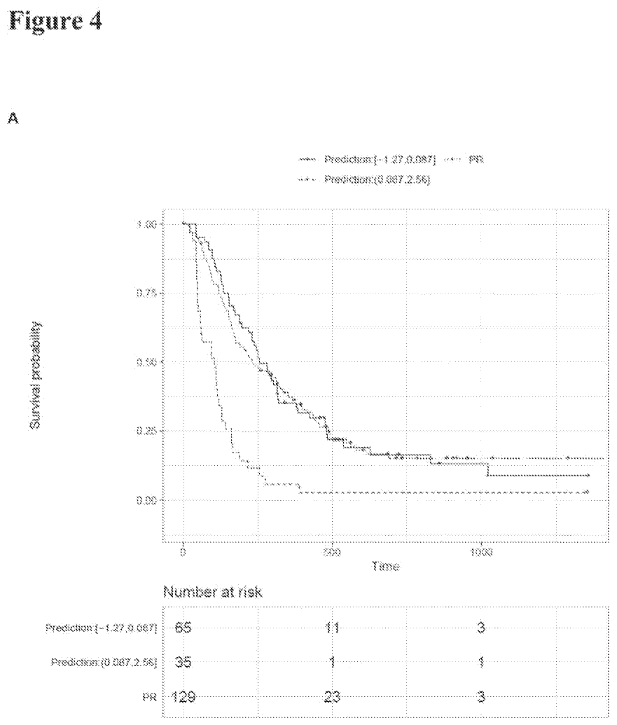

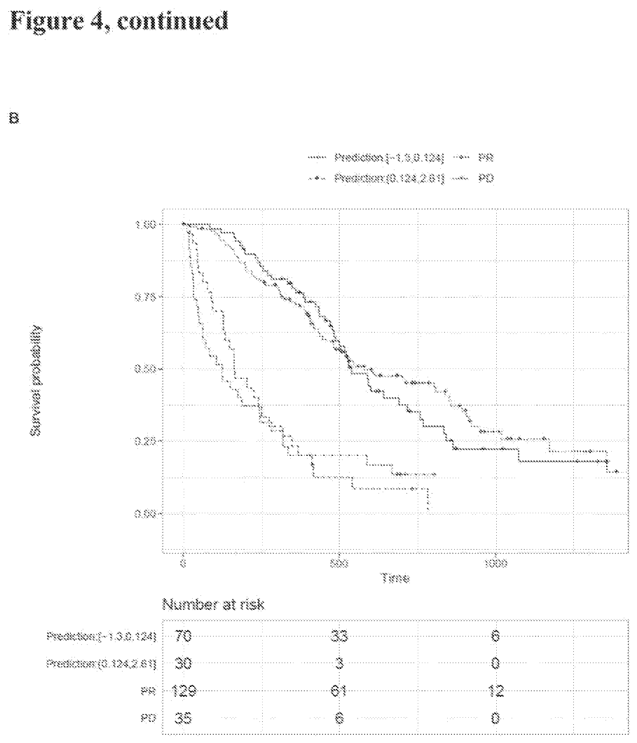

With the optimized cut-off, patients with high-risk SD according to the biomarker model had a similar survival probability to those with PD for OS, and patients with low-risk SD according to the biomarker model had a similar survival probability to those with PR for PFS and OS. (FIG. 4). Cut-off optimization was able to further increase the IR for the high vs low risk groups underlining the improved prognostic differentiation that can be made by using the biomarker score.

For PFS, the HR for high versus low risk of the model including CYFRA 21-1, CA125, CEA and the interaction term amounted to 3.241. The HR for PR versus low risk amounted to 1.023 (FIG. 4A).

For OS, HR for high versus low risk of the model including CYFRA 21-1. CA125, CEA and the interaction term amounted to 4.206. The HR for PR versus low risk amounted to 0.924. The HR for PD versus high risk amounted to 0.940 (FIG. 4B).

### Analyses Based on Combination of Individual Biomarker Splits (Binary Score)

The analysis using the model/score based on CYFRA21-1 and CA125 using the optimized cutoff was repeated by applying the optimized cutoff (as described above) to the single biomarkers, separating all patients into a low-risk SD and high-risk SD group based on the single marker and then combining the splits of the two biomarkers. In a nutshell, a binary score was built comprising a cut-off for CYFRA21-1 and CA125, respectively, and the CYFRA21-1 level and CA125 level were compared to the respective cut-offs, respectively. If a biomarker level was higher than the cut-off, the respective part of the binary score was defined as high. If a biomarker level was below or at the cut-off, the respective part of the binary score was defined as low. The resulting groups of the binary score based on the combination of the individual splits were then further summarized in a group with high risk (CYFRA21-1 above cutoff and CA125 above cutoff) versus low risk (CYFRA21-1 below cutoff and CA125 below cutoff, CYFRA21-1 below cutoff and CA125 above cutoff, CYFRA21-1 above cutoff and CA125 below cutoff.

With the optimized cut-off, patients with high-risk SD according to the biomarker model had a similar survival probability to those with PD for OS, and patients with low-risk SD according to the biomarker model had a similar survival probability to those with PR for PFS and OS.

For PFS based on all patients, the HR for high versus low risk of the model including CYFRA 21-1 and CA125 term amounted to 3,430. The HR for PR versus low risk amounted to 0.856.

For OS based on all patients, the HR for high versus low risk of the model including CYFRA 21-1 and CA125 amounted to 3.341. The HR for PR versus low risk amounted to 0.796. The HR for PD versus high risk amounted to 0.913. Accordingly, the binary score delivers similar results as a score using linear combination.

c) Prognostic Value of Cancer Biomarkers Using Baseline Correction of Biomarker Levels with the Biomarker Levels Before Treatment.

The results obtained by analyses using baseline corrected biomarker levels (i.e. ratio of the biomarker levels at first CT after second treatment cycle and initial biomarker concentration before treatment) for progression free survival are summarized in Table 6 below. The baseline corrected biomarker levels are log 2 transformed.

TABLE 6
Results of univariate and combination analyses of the prognostic values of the ratio
of CYFRA 21-1, CA 125 and CEA to BL for progression free survival in patients with
stable disease at the first CT scan after the second cycle. Higher C-index indicates
better performance of the Cox regression model, higher HR indicates better performance
in separating the SD population in high and low risk populations.

| | | | | | | | Adeno + SCC with | |
|---|---|---|---|---|---|---|---|---|
| | | | | | Adeno + SCC | | interaction term | |
| | Adeno (n = 76) | | SCC (n = 24) | | (n = 100) | | (n = 100) | |
| | C- | HR | C- | HR | C- | HR | C- | HR |
| | index | median^{a} | index | median^{a} | Index | median^{a} | index | median^{a} |
| Univariate | | | | | | | | |
| analysis | | | | | | | | |
| CYFRA 21-1 | 0.461 | 0.830 | 0.454 | 1.617 | 0.522 | 0.785 | 0.482 | 1.057 |
| CA 125 | 0.513 | 0.907 | 0.610 | 2.749 | 0.535 | 1.115 | 0.538 | 0.979 |
| CEA | 0.596 | 2.086 | 0.487 | 1.076 | 0.576 | 1.786 | 0.581 | 1.624 |
| Combination | | | | | | | | |
| analysis | | | | | | | | |
| CYFRA 21-1 + | 0.510 | 0.777 | 0.613 | 2.749 | 0.553 | 1.092 | 0.538 | 0.945 |
| CA 125 | | | | | | | | |
| CYFRA 21-1 + | 0.602 | 2.102 | 0.617 | 2.816 | 0.576 | 1.486 | 0.597 | 1.907 |
| CA 125 + CEA | | | | | | | | |

^{a}Patients stratified according to biomarker value above or below median.
Adeno, adenocarcinoma;; HR, hazard ratio; SCC, squamous cell carcinoma.

The results surprisingly show that the data without baseline correction relying on the absolute level of the biomarker (see table 2) show much better results in differentiating the SD patient group into high and low risk patients for PFS and OS than using the biomarker level changes (see table 6). It is a true advantage and a surprising finding that the risk assessment and, thus, the decision whether the patient benefits from the treatment can be based on a single biomarker measurement and does not require a baseline measurement. Even more surprising is that the performance of single biomarker measurements and scores derived therefrom is by far superior compared to the same biomarkers yet based on a change in the biomarker(s) level(s) compared to baseline (before treatment).

### CONCLUSIONS

CT is currently the method of choice for assessing response to therapy in lung cancer; however, in this study patients with PR at the first CT scan after the second treatment cycle had a similar risk of progression and survival to patients with SD. Furthermore, CT scan and the categorical RECIST criteria do not permit risk discrimination within patients with SD.

Of 7 biomarkers tested, the optimal prognostic biomarker differed depending on histology of the tumor; CYFRA 21-1 had the highest prognostic value for adenocarcinoma NSCLC, and CA 125 for SCC NSCLC.

Patients with SD could be separated into two prognostic groups based on the CYFRA 21-1, CA 125 and CEA combination (score using an interaction term taking the histology associated performance of the biomarkers CYFRA 21-1 and CA 125 and optionally CEA into account), above or below median; high-risk and low-risk. The prognostic performance of the triple combination (score using an interaction term taking the histology dependent performance of the biomarkers CYFRA 21-1 and CA 125 and optionally CEA into account) increased when patients were separated based on the optimized cut-off, suggesting that patients with high-risk SD outcome are comparable to those with PD for OS, and patients with low-risk SD have outcome comparable to those with PR for OS and PFS.

We have demonstrated herein that CYFRA 21-1 and CA 125 absolute levels as such, a score combining the absolute levels of the two biomarkers and in particular a score taking into account CYFRA 21-1. CA 125 and CEA absolute levels can provide additional guidance to the first CT scan after the second treatment cycle in patients with indeterminate CT response, by differentiating those with SD into high and low risk groups for PFS and OS. Further, we have demonstrated that the biomarker score can be further improved by taking into account an interaction term based on tumor histology (SCC or adenocarcinoma) and optimizing the cut-off.

A particular surprising finding of this study was that the biomarker levels based on a single measurement after the first CT scan after the second treatment cycle showed a much better performance in the stratification of SD patients being at high risk for disease progression and SD patients being at low risk of disease progression than changes in the levels of the same biomarkers measured before treatment. This finding indicates that a single biomarker measurement without the cumbersome requirement of continuous monitoring of biomarker levels is sufficient and even best for predicting disease progression. Furthermore, the sufficiency of only a single measurement will open up this risk assessment also to patients that have not had a continuous biomarker survey and avoids any potential problems with usage of different methods for the measurements at different time points of the treatment.

These findings can help physicians to identify patients who are more or less likely to respond to therapy early (after Cycle 2). The ability to predict already at the time of the first CT scan in patients with SD may offer additional guidance in stable disease and thus indeterminate radiological results and help guiding further treatment decisions (e.g. monitoring for possible treatment adjustments).

1. An in vitro method for assessing the risk of non-small cell lung carcinoma (NSCLC) disease progression for a subject suffering from non-small cell lung carcinoma (NSCLC) and being under an ongoing NSCLC treatment regime, wherein the subject is classified to have a stable disease, and wherein said method comprises:
a) determining a level of CYFRA 21-1 and/or a level of CA 125 in a sample obtained from the subject; and
b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score.

2. The method of claim 1, wherein in step (a) the level of CYFRA 21-1 is determined and the comparing in step (b) comprises (i) as defined in claim 1(b),
wherein a determined level of CYFRA 21-1 lower than or equal to the CYFRA 21-1 cut-off level is indicative of a low risk of NSCLC disease progression; and/or
wherein a determined level of CYFRA 21-1 higher than the CYFRA 21-1 cut-off level is indicative of a high risk of NSCLC disease progression.

3. The method of claim 2, wherein the subject suffers from NSCLC of subtype adenocarcinoma (ADC-NSCLC).

4. The method of claim 1, wherein in step (a) the level of CA 125 is determined and the comparing in step (b) comprises (ii) as defined in claim 1(b),
wherein a determined level of CA 125 lower than the CA 125 cut-off level is indicative of a low risk of NSCLC disease progression; and/or
wherein a determined level of CA 125 higher than the CA 125 cut-off level is indicative of a high risk of NSCLC disease progression.

5. The method of claim 4, wherein the subject suffers from NSCLC of subtype squamous cell carcinoma (SCC-NSCLC).

6. The method of claim 1, wherein in step (a) the levels of CYFRA 21-1 and/or CA 125 are determined and the comparing in step (b) comprises (iii) as defined in claim 1(b), and wherein a determined score lower than the cut-off score is indicative of a low risk of NSCLC disease progression; and/or
wherein a determined score higher than the cut-off score is indicative of a high risk of NSCLC disease progression.

7. An in vitro method for assessing whether for a subject diagnosed with non-small cell lung carcinoma (NSCLC) an ongoing NSCLC treatment regime is to be maintained or modified and/or whether a subject diagnosed with non-small cell lung carcinoma (NSCLC) responds to an ongoing NSCLC treatment regime, wherein said subject is classified to have a stable disease, and wherein said method comprises:
a) determining a level of CYFRA 21-1 and/or a level of CA 125 in a sample obtained from the subject; and
b) comparing (i) the determined level of CYFRA 21-1 to a CYFRA 21-1 cut-off level, (ii) the determined level of CA 125 to a CA 125 cut-off level, or (iii) a score taking into account the determined level of CYFRA 21-1 and/or the determined level of CA 125 to a cut-off score.

8. (canceled)

9. The method of claim 1, wherein the method further comprises determining a level of CEA, and wherein the comparing in step (b) comprises (iii) as defined in claim 1(b), and wherein the score further takes into account the determined level of CEA.

10. The method of claim 1, wherein the method further comprises:
obtaining an information whether the subject suffers from non-small cell lung carcinoma subtype squamous cell carcinoma (SCC-NSCLC) or adenocarcinoma (ADC-NSCLC);
wherein the comparing in step (b) comprises (iii) as defined in claim 1(b), and wherein the score further takes into account the NSCLC subtype.

11. The method of claim 1, wherein the sample is a sample obtained 10 days to 150 days after the start date of the ongoing treatment regime.

12. The method of claim 1, wherein the sample is a blood sample.

13. (canceled)

14. (canceled)

15. (canceled)

16. (canceled)

17. A kit comprising:
a reagent or a set of reagents for detecting a level of CYFRA 21-1 and/or a reagent or a set of reagents for detecting a level of CA 125, and optionally a reagent or set of reagents for detecting a level of CEA in a sample obtained from a subject,
wherein the kit is a kit for assessing:
(i) the risk of NSCLC disease progression under an ongoing NSCLC treatment regime for a subject diagnosed with NSCLC;
(ii) whether a subject diagnosed with NSCLC responds to an ongoing NSCLC treatment regime; and/or
(iii) whether for a subject diagnosed with NSCLC an ongoing NSCLC treatment regime is to be maintained or modified;
wherein said subject of (i), (ii), and (iii) is classified to have a stable disease.

18. The method of claim 7, wherein the method further comprises determining a level of CEA, and wherein the comparing in step (b) comprises (iii) as defined in claim 7(b), and wherein the score further takes into account the determined level of CEA.

19. The method of claim 7, wherein the method further comprises:
obtaining an information whether the subject suffers from non-small cell lung carcinoma subtype squamous cell carcinoma (SCC-NSCLC) or adenocarcinoma (ADC-NSCLC);
wherein the comparing in step (b) comprises (iii) as defined in claim 7(b), and wherein the score further takes into account the NSCLC subtype.

20. The method of claim 10, wherein the score further takes into account the NSCLC subtype by using an interaction term between the information about the NSCLC subtype and the level of CYFRA 21 and/or the level of CA 125.

21. The method of claim 11, wherein the sample is a sample obtained 20 days to 120 days after the start date of the ongoing treatment regime.

22. The method of claim 11, wherein the sample is a sample obtained 25 days to 108 days after the start date of the ongoing treatment regime.

23. The method of claim 12, wherein the sample is a blood sample selected from the group consisting of whole blood, serum, and plasma.

24. The method of claim 1, wherein said subject is a subject classified to have a stable disease based on imaging data.

25. The method of claim 1, wherein the ongoing NSCLC treatment regime is selected from chemotherapy, targeted therapy, and immunotherapy, and combinations thereof.
